(19) United States
(12) Patent Application Publication
(10) Pub. No.: US 2003/0024001 A1
(43) Pub. Date: Jan. 30, 2003

(54) Knock in transgenic mammal containing a non-functional N-terminus of Kv beta 1.1 subunit

(75) Inventors: Seung P. Kwak, Princeton, NJ (US); Kenneth Rhodes, Hillsborough, NJ (US); Karen L. Marquis, Yardley, PA (US); Thomas A. Comery, Langhorne, PA (US); Roger Askew, Boxford, MA (US); Michael Brandt, Flemington, NJ (US); Sharon Rosenzweig-Lipson, East Brunswick, NJ (US)

Correspondence Address: Bill T. Brazil, Five Giralda Farms, Madison, NJ, 07940-0874, US

(73) Assignee: Wyeth, Madison, NJ (US)

(21) Appl. No.: 10/196,890

(22) Filed: Jul. 16, 2002

Related U.S. Application Data
(60) Provisional application No. 60/308,485, filed on Jul. 27, 2001.
(60) Provisional application No. 60/331,140, filed on Nov. 9, 2001.

(51) Int. Cl. A01K 67/027

(52) U.S. Cl. 800/18; 800/14

(57) ABSTRACT

This invention provides a transgenic mammal containing a defective beta 1 subunit (Kvβ1) of a voltage sensitive potassium channel, where the Kvβ1 subunit is unable to confer N-type inactivation of the K⁺ but retains the ability to co-associate with Kv1 family α-subunits and thereby enhance channel surface expression. Preferably the Kvβ1.1 gene encoding Kvβ1 subunit has a mutation in all or a portion of codons 1-70 of its inactivation domain. The transgenic mammal is useful as a model for psychiatric and neurological disorders to identify anxiolytic compounds and pro-cognitive functions. The invention also provides for methods for screening and evaluating test compounds for their ability to modulate Kvβ1.1 activity, specifically for inactivation of a potassium channel or for co-association with α-subunits.

This application claims priority from a copending provisional application serial No. 60/308,485, filed on Jul. 27, 2001, the entire disclosure of which is hereby incorporated by reference.

### FIELD OF THE INVENTION

This invention is directed to a transgenic mammal containing a defective voltage sensitive potassium channel beta 1 subunit (Kvβ1), wherein the Kvβ1 subunit is unable to confer N-type inactivation of the channel but retains the ability to co-associate with Kv1 family α-subunits and thereby enhance channel surface expression. The transgenic mammal is useful as a model for psychiatric and neurological disorders and to identify anxiolytic compounds.

### BACKGROUND OF THE INVENTION

Voltage-gated potassium channels (Kv) contribute to nervous excitability in mammals. Some K⁺ channels (A-type K⁺ channels) are fast-inactivators which act as regulators for neuronal firing due to their fast inactivation. The discrete localization of Kv channels in brain tissue suggests that these channels are essential elements in the control of action potential propagation and neurotransmitter release in pathways frequently associated with seizure propagation and ischemic insult (see Rhodes et al. (1997) J. Neurosci. 17:8245-8258).

Some Kv channels are members of the Shaker-related superfamily, and are assembled from membrane-integrated aαsubunits and auxiliary cytoplasmic β subunits. Three Kvβ genes, termed Kvβ1, Kvβ2 and Kvβ3 provide five β subunits. Moreover, alternative splicing of the Kvβ1 gene provides three tissue-specific β1 subunit isoforms, Kvβ1.1, Kvβ1.2 and Kvβ1.3, which differ in their N-terminal sequences and in their expression patterns. Kvβ1.2 and Kvβ1.3 are expressed in heart tissue, while Kvβ1.1 is expressed in the brain tissue, particularly in the hippocampal CA1 region and striatum.

The β-subunits of the Kv channels play important roles in regulating the surface expression, stability, post-translational processing, and inactivation kinetics of the pore-forming α-subunits. Co-expression of any of the auxiliary Kvβ1 subunits with certain α subunits have resulted in a dramatic acceleration of the inactivation kinetics of the expressed Kv channels (Rettig et al. (1994) Nature 369:289-294; Majumder et al. (1995) FEBS Lett. 361, Morales et al. (1995) J. Biol. Chem. 270:6272-6277; England et al. (1995) Proc. Natl. Acad. Sci. USA 92:6309-6313; England et al. (1995) J. Biol. Chem. 270:28531-28534;: 13-16; McCormack et al. (1995) FEBS Lett. 370:32-36; Heinemann et al. (1996) J. Physiol. (Loud.) 493:625-633). It has previously been shown that inactivation of Kv channels is conferred by a “ball” domain in the amino-termini of Kvβ1 subunits (Rettig, supra). In addition, it has been suggested that the Kvβ1.1 subunits have a chaperone-like function on the folding of corresponding Kv1 family α-subunits, since they promote surface expression of coexpressed α subunits (Shi et al. (1996) Neuron 16:843-852; Accili et al. (1997) J. Biol. Chem. 272:25824-25831). The ability of the Kvβ1 subunits to co-associate with corresponding Kv1 family α-subunits is retained despite deletion of their amino-termini and consequently, there is a loss of the ability to confer channel inactivation, as shown in experiments with transfected mammalian cells (Nakahira et al. (1996) J. Biol. Chem. 271:7084-7089). Significantly, complete loss of function of the entire Kvβ1.1 subunits leads to a reduction of A-type Kv channel activity in hippocampal and striatal neurons of knock-out mice (Giese et al. (1998) Learning & Memory 5:257-273). It is unclear however, how the functionality of Kvβ1.1 subunits, whether it be N-type inactivation or their chaperone-like function, contributes to altered cognition, anxiety and motor control of an animal.

PCT publication WO 00/24871 discloses a transgenic mouse having a knock-out mutation disabling the whole of the Kvβ1.1 subunit of the A-type K+ channel. WO 00/24871 does not describe however, a transgenic mammal having a mutation wherein only a portion of the Kvβ1.1 subunit has been inactivated to leave portions of the Kvβ1.1 subunit which retain functionality. In particular, WO 00/24871 does not provide a transgenic animal having a mutation of the Kvβ1.1 subunit wherein all functional properties of β1.1 subunit are retained except the ability to confer N-type inactivation.

It would be desirable to provide a transgenic mammalian model wherein distinct portions of the Kvβ1.1 subunit have been rendered non-functional, in particular, wherein N-type inactivation has been lost, so as to identify compounds (“disinactivators”) and therapies which may be useful in the treatment of panic and anxiety disorders, cognitive disorders, epilepsy, ischemic stroke and movement disorders.

### SUMMARY OF THE INVENTION

The present invention is directed to a transgenic rodent having an endogenous gene cluster which encodes a mutated Kvβ1.1 subunit in an A-type potassium channel, wherein the mutated Kvβ1.1 subunit is a knock-in subunit which is unable to confer N-type inactivation of the channel but retains the ability to co-associate with Kv1 family α-subunits.

In a preferred embodiment, the transgenic rodent is a mouse (“KI mouse”) and the mutated knock-in Kvβ1.1 subunit is encoded by a homozygous mutation. The knock-in Kvβ1.1 subunit may be encoded by a mutation selected from the group consisting of replacement mutations, insertion mutations, frameshift mutations and stop codon mutations, most preferably the KI mouse manifests a significantly different learning or memory pattern as compared to a mouse wherein the Kvβ1.1 subunit is completely non-functional (“KO mouse”), as assayed by a Y maze. In one aspect, the Y maze test will preferably indicate that the KI mouse has significantly improved learning or memory after a 4 hour inter-trial interval as compared to the KO mouse, but that the KI mouse has significantly impaired learning or memory after a 30 minute inter-trial as compared to a KO mouse. In another preferred embodiment, a contextual fear conditioning assay is used to indicate that the KI mouse has a significantly impaired learning pattern as compared to a KO mouse and to a mouse of the same strain having a wild-type Kvβ1.1 subunit (“WT mouse”). In yet another preferred embodiment, an elevated zero maze assay is used to indicate that the KI mouse has a significantly reduced anxiety pattern as compared to a KO and a WT mouse. In still another aspect, an assay for stress-induced corticosterone levels is used to indicate that the KI mouse has a significantly reduced anxiety pattern as compared to a KO and a WT mouse. Similarly, in another aspect of the invention, an assay for stress-induced hyperthermia is used to indicate that the KI mouse has a significantly reduced anxiety pattern as compared to a KO and a WT mouse.

The present invention is also directed to a transgenic rodent whose genome comprises a homozygous mutation in codons 1-70 (i.e., SEQ ID NO: 1) of the N-terminus of an endogenous Kvβ1.1 subunit gene, preferably codons 1-3, where said mutation is a replacement mutation and the rodent exhibits significantly different cognitive patterns over a second rodent whose genome comprises a homozygous mutation which encodes a completely non-functional Kvβ1.1 subunit. Preferably, the replacement mutation comprises an immunoreactive epitope tag, and most preferably, the replacement mutation is a hemagglutinin epitope tag. In a further aspect of the invention, the transgenic rodent is a mouse.

In another embodiment, the present invention is directed to a transgenic rodent whose germ cells and somatic cells contain a recombinant activated Kvβ1.1 transgene sequence introduced into said rodent, or an ancestor of said rodent, at an embryonic stage, where the Kvβ1.1 transgene encodes a β subunit which is unable to confer N-type inactivation of a potassium channel but retains the ability to co-associate with Kv1 family α-subunits.

In another aspect, the present invention provides a method of making an isolated knock-in mammalian cell comprising the steps of (1) effecting homologous recombination between an endogenous Kvβ1.1 gene and a transgene Kvβ1.1, wherein said transgene Kvβ1.1 comprises (a) a sequence encoding an immunoreactive tag substituting all or a portion of codons 1-70 of the Kvβ1.1 subunit, (b) a selectable marker flanked by a pair of repeat sites, and (c) a pair of sequences homologous to the endogenous Kvβ1.1 gene flanking both the tag and the selectable marker; and, (2) effecting further recombination to remove the selectable marker, wherein the transgene Kvβ1.1 encodes a β subunit which is unable to confer N-type inactivation but retains the ability to co-associate with Kv1 family α-subunits. Preferably, the mammalian cell is homozygous for the transgene. Most preferably, the immunoreactive tag substitutes all or a portion of codons 1-36.

In yet another embodiment, the present invention provides a mammalian cell expressing an A-type potassium channel having a knock-in Kvβ1.1 subunit wherein the knock-in Kvβ1.1 subunit is unable to confer N-type inactivation of the channel but retains the ability to co-associate with Kv1 family α-subunits, where said cell comprises an endogenous nucleic acid sequence which controls expression of the knock-in Kvβ1.1 subunit and said knock-in Kvβ1.1 subunit is encoded by a mutation selected from the group consisting of a replacement mutation, an insertion mutation, a frameshift mutation, and a stop codon mutation. Preferably, the cell is selected from the group consisting of a horse, bovine, rodent, cat, dog, pig, goat, sheep, non-human primate, human, rabbit and hamster. Most preferably, the cell is a murine cell. In another aspect, the cell comprises a mutation in which all or a portion of codons 1-70 in the endogenous nucleic acid sequence are replaced, more preferably codons 1-36 are replaced, and the replacement comprises an immunoreactive epitope tag. Most preferably, the cell is homozygous for the replacement mutation.

In a further embodiment, the present invention provides a nucleic acid construct encoding a mutation in codons 1-70 of a Kvβ1.1 gene, more preferably in codons 1-36; wherein said nucleic acid encodes a knock-in subunit of an A-type potassium channel and said nucleic acid knock-in subunit is unable to confer N-type inactivation of the A-type potassium channel but retains the ability to co-associate with Kv1 family α-subunits. The mutation may be a replacement mutation, an insertion mutation, a frameshift mutation, and a stop codon mutation, but is preferably a replacement mutation. Most preferably, the mutation comprises an immunoreactive epitope tag replacing all of codons 1-70, particularly codons 1-36, of the Kvβ1.1 gene. Furthermore, the nucleic acid construct comprises nucleic acid which is either deoxyribonucleic acid (DNA) or is ribonucleic acid (RNA). In another aspect, the nucleic acid construct is in either a mammalian cell or a vector.

In another aspect, the present invention provides a nucleic acid construct for disrupting expression of an endogenous Kvβ1.1 gene via homologous recombination, where said construct comprises an immunoreactive epitope tag replacing all, or a portion of, codons 1-70 of the Kvβ1.1 gene, a selectable marker and a pair of nucleic acid sequences flanking both the tag and the selectable marker, where the pair is homologous to a portion of the endogenous Kvβ1.1 gene. More preferably, the immunoreactive epitope tag replaces all or a portion of codons 1-36. Preferably, the immunoreactive epitope tag is a hemagglutinin epitope tag or the selectable marker is a neo gene. More preferably, the selectable marker is further flanked by Lox P nucleic acid sequences. Most preferably, the nucleic acid construct is in a vector.

In yet another embodiment, the present invention provides a method of pre-screening test compounds for modulators of Kvβ1.1 subunit activity, comprising the steps of (a) contacting test compounds with a knock-in Kvβ1.1 subunit; and (b) selecting one of the test compounds which provides a detectable change in the activity of the knock-in Kvβ1.1 subunit, where the knock-in Kvβ1.1 subunit is unable to confer N-type inactivation but retains the ability to co-associate with Kv1 family α-subunits. In one embodiment, the knock-in Kvβ1.1 subunit is in a test sample, said test sample comprises a cell, and the step of contacting comprises administering said test compound to the cell. In addition, the step of detecting may comprise using an immunoassay to determine whether the Kvβ1.1 subunit co-associates with Kv1 family α-subunits. In one embodiment, the selected test compound binds to the Kvβ1.1 subunit. Preferably, the test compounds are small molecules selected from a group of libraries consisting of spatially addressable parallel solid phase or solution phase libraries or synthetic libraries made from deconvolution, “one-bead one-compound” methods or by affinity chromatography selection.

Alternatively, the present invention provides a method of pre-screening test compounds for modulators of Kvβ1.1 subunit activity by (a) contacting the test compounds with a wild-type Kvβ1.1 subunit and a knock-in Kvβ1.1 subunit; and (b) selecting one of the test compounds which provides a detectable change in the activity of the wild-type Kvβ1.1 subunit but no detectable change in the activity of the knock-in Kvβ1.1 subunit, where the knock-in Kvβ1.1 subunit is unable to confer N-type inactivation but retains the ability to co-associate with Kv1 family α-subunits. In one embodiment, the wild-type and knock-in Kvβ1.1 subunits are in one test sample. Alternatively, the wild-type Kvβ1.1 subunit is in a first test sample and the knock-in Kvβ1.1 subunit is in a second test sample. The test samples may comprise cells, in which case, the step of contacting comprises administering said test compound to the cells. In one embodiment, a Kv1 family α-subunit is also present with the test compounds and an immunoassay may be used to determine whether the detectable change is due to a lack of co-association with Kv1 family α-subunits. In one embodiment, the selected test compound binds to the Kvβ1.1 subunit. In addition, as described above, the test compounds may be small molecules selected from a group of libraries consisting of spatially addressable parallel solid phase or solution phase libraries or synthetic libraries made from deconvolution, “one-bead one-compound methods” or by affinity chromatography selection.

In another preferred embodiment, the present invention provides a method of assessing the efficacy of a test compound for modulating the activity of a Kvβ1.1 subunit by contacting the test compound with a wild-type Kvβ1.1 subunit and a mutant Kvβ1.1 subunit, and detecting a change in activity of the wild-type Kvβ1.1 subunit but no change in activity of the knock-in Kvβ1.1 subunit in the second test sample, where the knock-in Kvβ1.1 subunit is unable to confer N-type inactivation of a potassium channel but co-associates with Kv1 family α-subunits. Preferably, the test compound reduces the activity of the wild-type Kvβ1.1 subunit by greater than 10%, more preferably by greater than 50%. In one embodiment, the wild-type Kvβ1.1 subunit is in a first test sample and the knock-in Kvβ1.1 subunit is in a second test sample. The test sample may comprise either a cell, a tissue or a transgenic rodent. Preferably, the step of contacting comprises administering said test compound to the rodent, and more preferably, the rodent is a mouse.

In alternative preferred embodiments, the step of contacting comprises contacting the test compound with a tissue or a cell. In a further preferred embodiment, the method comprises the step of contacting the test compound with a third test sample and detecting no change in activity of the completely non-functional Kvβ1.1 subunit in the third test sample, where the third test sample is characterized by expression of a completely non-functional Kvβ1.1 subunit. Moreover, where the step of contacting comprises contacting the test compound with a cell, the step of detecting comprises an immunoassay to determine whether the wild-type Kvβ1.1 subunit co-associates with Kv1 family α-subunits. More preferably, the knock-out subunit is in a rodent and the step of detecting in this embodiment comprises a behavioral test such as a Y maze, contextual fear conditioning and an elevated zero maze. Alternatively, the knock-out subunit is in a rodent, and the step of detecting in this embodiment comprises a physiological assay such as a hormonal assay, a hyperthermia assay and an electro-physiological assay.

In still another embodiment, the present invention includes a method of assessing the efficacy of a test compound for inactivating A-type potassium channels by contacting a test compound with a wild-type Kvβ1.1 subunit and a knock-in Kvβ1.1 subunit and detecting a change in the activity of the wild-type Kvβ1.1 subunit but no change in the activity of the knock-in Kvβ1.1 subunit, where the knock-in Kvβ1.1 subunit is encoded by a Kvβ1.1 gene sequence comprising a mutation in all, or a portion of, codons 1-70, more preferably codons 1-36. In one embodiment, the change in the activity of the wild-type Kvβ1.1 subunit is caused by inhibition of N-type inactivation of the potassium channels. Preferably, the change in the activity of the wild-type Kvβ1.1 subunit causes the wild-type subunit to have the same activity as the knock-in Kvβ1.1 subunit. In another preferred embodiment, the activity is measured by subjecting a transgenic rodent to an elevated zero maze. Alternatively, the activity is measured by subjecting a transgenic rodent to an environmental stimulus and then measuring the corticosterone levels of the rodent. In another alternative embodiment, the activity is measured by taking tissue from a transgenic rodent and subjecting the tissue to electro-physiological tests. Most preferably, the transgenic mammal is a mouse. In yet another alternative embodiment, the activity is measured by using in vitro binding assays. Moreover, in another embodiment, the test compound binds to the wild-type Kvβ1.1 subunit but does not bind to the knock-in Kvβ1.1 subunit. In a most preferred embodiment, the method further comprises contacting the test compound to a completely non-functional Kvβ1.1 subunit and detecting no change in the activity of the completely non-functional Kvβ1.1 subunit. Preferably in this embodiment, the test compound reduces the activity of the wild-type Kvβ1.1 subunit by greater than 10%, and more preferably by greater than 50%.

### BRIEF DESCRIPTION OF THE DRAWINGS

FIG. 1A is a schematic representation of endogenous Kvβ1 gene structure and its exonic organization. The three exons at the 5′ end (exon 1.1, 1.2, 1.3) share common domains (exons 3-15) at the C-terminus but are alternately spliced so each encodes unique N-terminal protein sequences. Exon 1.1 encodes the inactivation domain that was targeted for mutation. The relative order of the three exons 1.1, 1.2 and 1.3 on the gene is uncertain.

FIG. 1B illustrates the strategy by which the endogenous Kvβ1.1 gene was targeted for mutation. The targeting vector (top) was designed to incorporate a 3×HA tag in place of the 33^{rd} codon of exon 1.1 and a lox-flanked neomycin cassette (Flox-Neo). Through homologous recombination (first arrow), the mutant exon and Flox-Neo is exchanged in place of exon 1.1. A second recombination mediated by Cre-recombinase (second arrow) removes the Flox-Neo from the intron, leaving behind one copy of the LoxP site (bottom). The restriction sites EcoRI and Bam HI are represented by “R” and “B”, respectively.

FIG. 1C is a diagrammatic representation of a Southern blot in which ES cells were digested with Bam HI. The 5′ probe from FIG. 1B was used to identify a ˜7.0 kb band for the wild-type Kvβ1 allele, while an additional ˜1.0 kb band, resulting from the BamHI site located within 3×HA, was identified in the three targeted ES clones (51, 145 and 191).

FIGS. 1D and 1E are a diagrammatic representation of the results of RNase protection analysis, indicating the expression pattern of knock-in Kvβ mutant (Kvβo) mRNA (FIG. 1D) and wildtype Kvβ (WT) (FIG. 1E). A probe complementary to 3×HA exon 1 generated a protected band of 420 bp in heterozygous and homozygous KI animals (wt/KI and KI/KI respectively) and a shorter protected band of 220 bp in wildtype (WT), due to a missing 3×HA sequence at the N-terminus of normal kvβ1.1 mRNA. Converse RPA analysis, using a probe complementary to normal Kvβ1.1 mRNA, demonstrated a fully protected fragment of 480 bp from WT mRNA, while two fragments (210 and 140) flanking the 3×HA sequence were protected in KI mRNA. Animals bearing the Flox-Neo cassette did not show specific bands from either probes, indicating complete deficiency of Kvβ1.1 expression and a KO genotype.

FIG. 1F is a diagrammatic representation of the results of PCR analysis using samples from various genotypes. Kvβo KI primers amplify a fragment between the 3′exon-intron boundary of exon 1.1 and a site 30 bp downstream of Neo-Flox, and thereby permits identification of homozygous wild-type (w/w), homozygous KO (o/o), homozygous KI (I/I) and heterozygous KI (w/I) animals. The KI allele includes the presence of a loxP repeat sequence and therefore provides a longer amplified sequence (264 bp) than the WT allele (230 bp).

FIG. 1G is a diagrammatic representation of a Western blot conducted to analyze expression of Kvβ1, Kvβ2, synapsin1 (control) or HA in cortex, striatum, hippocampus, cerebellum, midbrain and thalamus from WT and mutant mice. Brain regions as indicated were dissected and analyzed for specific immuno reactivities.

FIGS. 2A-2B illustrate different learning and memory patterns among WT 129/SvEv mice versus KO and KI mice in the Y maze (Example 2). FIG. 2A illustrates the percentage of mice which first choose the novel arm of the maze after inter-trial intervals (ITIs) of 30 min, 2 hr and 4 hr. FIG. 2B illustrates the percentage of time (of a 2 minute period) spent in the novel arm, after ITIs of 30 min, 2 hr and 4 hr.

FIGS. 3A-3B illustrate different learning patterns between WT, KI and KO mice in both C57BI6 (FIG. 3A) and 129/SvEv (FIG. 3B) backgrounds in the Contextual Fear Conditioning test.

FIGS. 4A-4B illustrates the percentage of time spent by the three different genotypes, WT, KI and KO, in both C57BI6 (FIG. 4A) and 129/SvEv (FIG. 4B) backgrounds, in the open zone portion of the elevated zero maze.

FIG. 5A illustrates the effect of varying dosages of chlordiazepoxide on stress-induced hyperthermia in male WT (129/SvEv) mice. Inset shows body temperature on the initial measurement.

FIG. 5B illustrates the reactions to stress-induced hyperthermia in wildtype (WT; 129/SvEv), Kvβ knock-in (KI) and knockout (KO) mice. Inset shows body temperature on the initial measurement.

FIG. 6 illustrates that seizure threshold is unchanged in Kvβ knock-in mice.

FIG. 7 illustrates in-situ hybridization quantitation of c-fos mRNA in the parietal cortex of KI, KO and wild-type mice.

### DETAILED DESCRIPTION OF THE INVENTION

The present invention is directed to the discovery that transgenic mice bearing a knock-in mutation (KI) in the Kvβ1.1 gene manifest significantly different behavioral phenotypes compared to transgenic mice bearing a knockout mutation (KO) of Kvβ1.1 or wild-type mice (WT). The β-subunits of KI mice lack the ability to inactivate Kv1-family K⁺ channels but retain the ability to co-associate with Kv1 family α-subunits and thereby enhance channel surface expression. In three different behavioral paradigms, the Y maze test, contextual fear conditioning and the elevated zero maze, KI mice consistently registered different behavioral patterns from KO mice. As indicated by the time spent in the novel arm of the Y maze (see Example 2), KI mice manifested retention deficits at the half hour inter-trial interval (ITI) as compared to KO and WT mice, but displayed improved retention over KO mice at the four hour ITI. In the contextual fear conditioning test, KI mice demonstrated significant impairments as compared to KO and WT mice (see Example 4). Furthermore, KI mice displayed different behavior and physiological patterns as compared to KO mice in functional assays such as the elevated zero maze (Example 4), stress-induced corticosterone levels (Example 5) and stress-induced hyperthermia (Example 6).

Without being bound by theory, it is believed that the KI mutation elicits a distinct phenotype from KO that reflects the specific deficit of N-type channel inactivation. The results of KI activity in behavioral and physiological assays suggest an anxiolytic profile, and therefore a potential role in screening test compounds for the treatment of panic and anxiety disorders.

The present invention is therefore directed to transgenic mammals, particularly mice, whose genome encodes the KI mutation and to the nucleic acid constructs and targeting constructs used in the generation thereof. In addition, the KI transgenic animals provide a positive model for evaluating the efficacy of test compounds that modulate Kvβ1.1 activity. The present invention is also directed to binding assays, high-throughput assays and functional assays (particularly behavioral and physiological assays) for test compounds capable of modulating Kvβ1.1 activity, as shown below.

### DEFINITIONS

To facilitate an understanding of the present invention, a number of terms and phrases are defined below:

The term Kvβ1.1 refers to a β1 subunit of an A-type potassium channel, in particular the β1.1 subunit isoform of a shaker-like voltage-gated potassium channel (Kv). The Kv channel is typically made up of four identical subunits, which join together around a central water-filled pore. This pore is opened to allow the passage of potassium ions through it, or closed in response to changes in cell potential; hence, “voltage-gated”. The Kv channels and the β1.1 subunit are known to those of skill in the art.

As used herein, a knock-in Kvβ1.1 mutation (“KI”) refers to a mutation in a Kvβ1.1 gene, whereby the mutated Kvβ1.1 gene encodes a defective β1.1 subunit which is unable to confer N-type inactivation of K⁺ channels but is able to co-associate with Kv1 family α-subunits and thereby enhance channel surface expression. As used herein, a KI subunit refers to the mutated β1.1 subunits encoded by the KI mutation, and a KI mammal refers to a mammal having an expressed KI mutation. In preferred embodiments, a KI mutation is generated by inducing a mutation in codons 1-70 of the Kvβ1.1 gene. Codons 1-70 of Kvβ1.1 are provided in SEQ ID NO: 1. The preferred DNA and amino acid sequence range of Kvβ1.1 is shown in SEQ ID NO: 1. Preferably, the mutation is a replacement mutation in which all, or a portion of, codons 1-36 are replaced, as shown in the “Kvβₒ” mutation of Example 1 and SEQ ID NO: 2. In particularly preferred embodiments, an immunoreactive tag substitutes all of endogenous codons 1-70, or at least 1-36, of the Kvβ1.1 gene and aids in detecting co-immuno-precipitation with an α-subunit. Alternatively, any foreign nucleic acid sequence may be inserted into the endogenous Kvβ1.1 gene to produce the KI mutation. A Kvβ1.1 knock-in mammal, or the tissues or cells therefrom, includes both the heterozygote mammal (i.e., one mutated allele and one wild-type allele) and the homozygous mutant (i.e., two mutated alleles).

A Kvβ1.1 knockout mutation (“KO”) refers to a mutation in a Kvβ1.1 gene, whereby the mutated Kvβ1.1 gene encodes a β-subunit which is unable to either confer N-type inactivation of the potassium channel or to co-associate with Kv1 family α-subunits, or wherein the Kvβ1.1 gene is completely absent. In short, the KO mutation encodes a completely non-functional Kvβ1 subunit. As used herein, a KO subunit refers to the mutated β1.1 subunits encoded by the KO mutation, and a KO mammal refers to a mammal having an expressed KO mutation. The term “Kvβ1.1-knockout” refers to both the heterozygote mammal and the homozygous mutant mammal, and the tissues or cells therefrom. An example of a Kvβ1.1-knockout mutation is shown in Example 1, wherein a neomycin marker is inserted into an intron of the Kvβ1.1 gene and completely disrupts functionality of the gene.

The term wild-type Kvβ1.1 (“WT”) refers to a Kvβ1.1 gene having naturally-occurring nucleotide and amino acid sequences encoding a completely functional Kvβ1.1 subunit, where the gene encoding the Kvβ1.1 subunit has not at any time, in immediate or ancestor generations, been experimentally manipulated to either delete naturally-occurring material or to include foreign genetic material.

The term “genetically modified” refers to a cell containing and/or expressing a gene comprising an induced mutation which in turn modifies the genotype and phenotype of the cell, and subsequently, its progeny. This term includes any addition or disruption to a cell's endogenous nucleotides, including an insertion mutation, a frameshift mutation or a stop codon mutation. The terms “mutated” or “mutant” and their various grammatical derivatives refers to any gene comprising an induced mutation, or to a gene product encoded therefrom. As used herein, a mutated Kvβ1.1 gene will typically refer to a gene which encodes either a knock-in Kvβ1.1 subunit or a knock-out Kvβ1.1 subunit.

The term “non-human mammals” of the invention includes any vertebrates such as rodents, non-human primates, ovines, bovines, ruminants, lagomorphs, porcines, caprines, equines, canines, felines, aves, etc. Preferred non-human mammals are selected from the order Rodentia which includes rats and mice, most preferably mice.

The term “transgene” as used herein refers to a foreign nucleic acid sequence that is placed into a subject mammal by introducing the foreign sequence into embryonic stem (ES) cells, newly fertilized eggs or early embryos. The term “foreign nucleic acid sequence” refers to any nucleic acid sequence which is introduced into the genome of an animal by experimental manipulations and may include nucleic acid sequences found in that mammal so long as the introduced gene contains some modification (e.g., an immunoreactive epitope tag, a point mutation, the presence of a selectable marker gene, the presence of a IoxP site, etc.) relative to the naturally-occurring gene.

The term “targeting construct” refers to an oligonucleotide sequence comprising a modification (e.g., an immunoreactive epitope tag, an insertion mutation, a stop codon mutation, the presence of a selectable marker gene, the presence of a IoxP site, etc), and a sequence(s) homologous to the endogenous Kvβ1 gene which flanks the modification. The targeting construct is generally ligated into a targeting vector, e.g., a plasmid, which is capable of introducing the construct into a host cell. The homologous sequence(s) permits the homologous recombination of the targeting construct or vector into at least one allele of the endogenous Kvβ1 gene in the chromosomes of the target or recipient cell (e.g., ES cells). The targeting construct or vector may contain more than one modification. Preferably, the targeting constructs and vectors of the present invention are of the “replacement-type,” where two regions of homology flank the gene modifications and result in the replacement of the portion of the targeted gene lying between the homologous regions. Insertion-type constructs and vectors by contrast, have only one region of homology with the targeted gene and results in the insertion of the adjacent portion into the targeted gene, typically at either the carboxy- or amino-terminus. If insertion-type constructs or vectors are used in the present invention, then the homologous region must target for insertion into the amino-terminus of the Kvβ1.1 gene. As demonstrated herein, homologous recombination permits the integration of targeting constructs and vectors to disrupt the inactivation domain of Kvβ1, resulting in the inability to confer N-type inactivation.

As used herein, the term “selectable marker” refers to a gene which encodes an enzymatic activity that confers resistance to an antibiotic or drug upon the cell in which the selectable marker is expressed. Selectable markers of the invention are preferably “positive”; positive selectable markers are typically dominant selectable markers, i.e. genes which encode an enzymatic activity which can be detected whenever present in a mammalian cell or cell line (including ES cells).

As used herein, the term “modulating activity”, in its various grammatical forms (e.g., “modulated,” “modulation,” “modulating,” etc.) includes, the stimulation, potentiation, inhibition and/or relief of inhibition of normal protein activity (e.g. wild-type Kvβ1.1 activity). The term does not encompass the up-regulation or down-regulation of expression levels of the protein.

The term “activity” as used in relation to a WT, KI or KO subunit, refers to the particular subunit's ability to carry on its normative functions, ie., normative WT subunit activity is both co-association with Kv1 family a subunits and inactivation of potassium channels, normative KI subunit activity is typically co-association with Kv1 family α subunits but no channel inactivation, and normative KO subunit activity is a complete inability to co-associate or inactivate potassium channels. A “detectable change in activity” or “detecting a change in activity” is therefore a deviation from the normative function of the particular subunit, as determined by the binding assays and functional assays described below. The binding assays of the invention typically use radiolabeling to detect binding, and therefore changes in activity, whereas the functional assays will use statistically significant deviations in behavior or physiology to detect changes in subunit activity.

The terms “polypeptide,” “peptide” and “protein” are used interchangeably herein to refer to a polymer of two or more subunit amino acids, amino acid analogs, or peptidomimetics. The subunit may be linked by peptide bonds or other bonds, e.g., ester, ether, etc. As used herein the term “amino acid” includes either natural and/or unnatural or synthetic amino acids, including glycine and both the D or L optical isomers, and amino acid analogs and peptidomimetics. A peptide of three or more amino acids is commonly referred to as an oligopeptide. Peptide chains of greater than three or more amino acids are referred to as a polypeptide or a protein.

As used herein, the terms “polynucleotide” and “oligonucleotide” are used interchangeably, and include polymeric forms of nucleotides of any length, either deoxyribonucleotides or ribonucleotides, or analogs thereof. Polynucleotides may have any three-dimensional structure, and may perform any function, known or unknown. The following are non-limiting examples of polynucleotides: a gene or gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term also includes both double- and single-stranded molecules. Unless otherwise specified or required, any embodiment of this invention that is a polynucleotide encompasses both the double-stranded form and each of two complementary single-stranded forms known or predicted to make up the double-stranded form.

An “antibody” includes an immunoglobulin molecule capable of binding an epitope present on an antigen. As used herein, the term encompasses not only intact immunoglobulin molecules such as monoclonal and polyclonal antibodies, but also anti-idiotypic antibodies, mutants, fragments, fusion proteins, bi-specific antibodies, humanized proteins, and modifications of the immunoglobulin molecule that comprises an antigen recognition site of the required specificity.

As used herein, the terms “binding partner,” or “capture agent,” or a member of a “binding pair” refers to molecules that specifically bind other molecules to form a binding complex such as antibody-antigen, lectin-carbohydrate, nucleic acid-nucleic acid, biotin-avidin, etc.

As used herein, the term “binds” or “specifically binds,” in relation to a biomolecule (e.g., small molecule, protein, nucleic acid, antibody, etc.), refers to a binding reaction which is determinative of the presence of the biomolecule in a heterogeneous population of molecules (e.g., proteins and other biologics). Thus, under designated conditions (e.g., immunoassay conditions in the case of an antibody or stringent hybridization conditions in the case of a nucleic acid), the specified ligand or antibody binds to its particular “target” molecule and does not bind in a significant amount to other molecules present in the sample.

The terms “mimic” or “imitate” when used in reference to the ability of a test compound to imitate the activity of a mutated Kvβ1.1 subunit, refers to the ability of the compound to produce effects (e.g. behavioral, electro-physiological, biochemical) substantially similar to those observed in mammals expressing a mutated Kvβ1.1 gene, i.e. KI or KO mutations.

The term “test compound” refers to an agent that is to be screened in one or more of the assays described herein. The agent can be virtually any chemical compound. It can exist as a single isolated compound or can be a member of a chemical (e.g., combinatorial) library. In a most preferred embodiment, the test compound is a small molecule.

The term “small molecule” refers to a molecule of a size comparable to organic molecules generally used in pharmaceuticals. The term excludes biological macromolecules (e.g., proteins, nucleic acids, etc.), but includes peptides, saccharides, fatty acids, steroids, purines, pyrimidines, derivatives, structural analogs or combinations thereof. Preferred small molecules range in size up to about 5000 Da, more preferably up to 2000 Da, and most preferably up to about 1000 Da. In addition, the small molecules may include numerous chemical classes, but are preferably organic molecules having functional groups which enable protein interaction, such as amines, carbonyls, hydroxyl or carboxyl groups. Typical small molecule compounds will include cyclical carbon or heterocyclic structures and/or aromatic or polyaromatic structures substituted with one or more of the above functional groups.

As used herein, a “test sample” refers to any sample suitable for the assays of the invention, including but not limited to cell samples, tissue samples and/or whole animals. Preferably the test sample is a biological sample obtained from an organism or from components (e.g., cells, tissue, fluid) of an organism.

Various aspects of the invention are described in further detail in the following subsections. The subsections below describe in more detail the present invention. The use of subsections is not meant to limit the invention; subsections may apply to any aspect of the invention.

I. Generation of Transgenic Kvβ1.1 Knock-in Mammals.

A. Design of the Targeting Construct

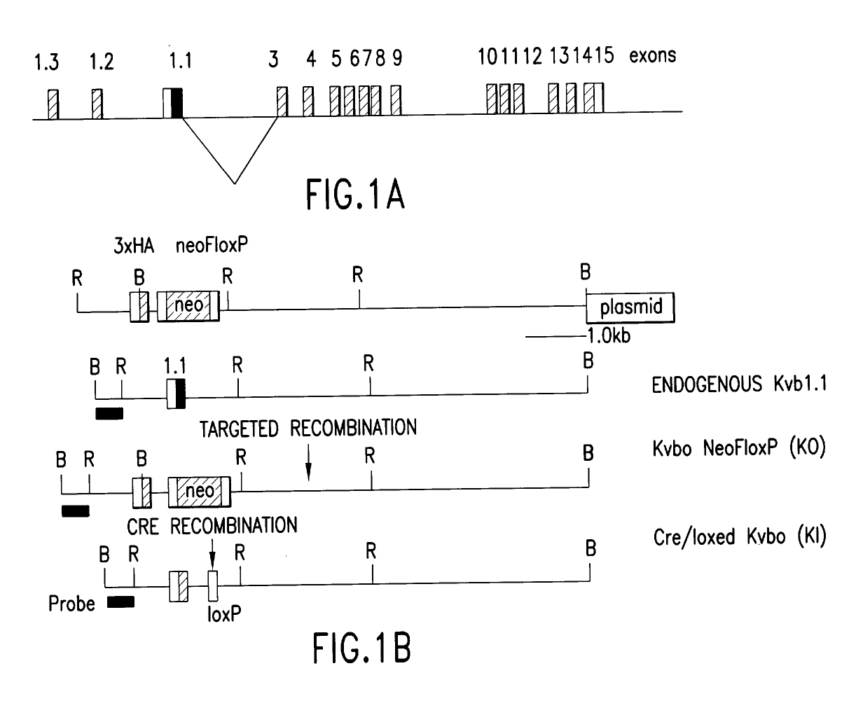

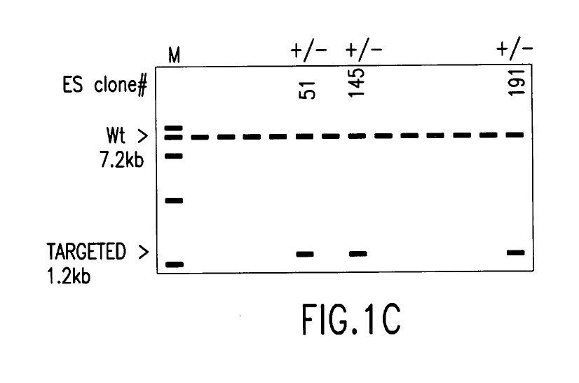

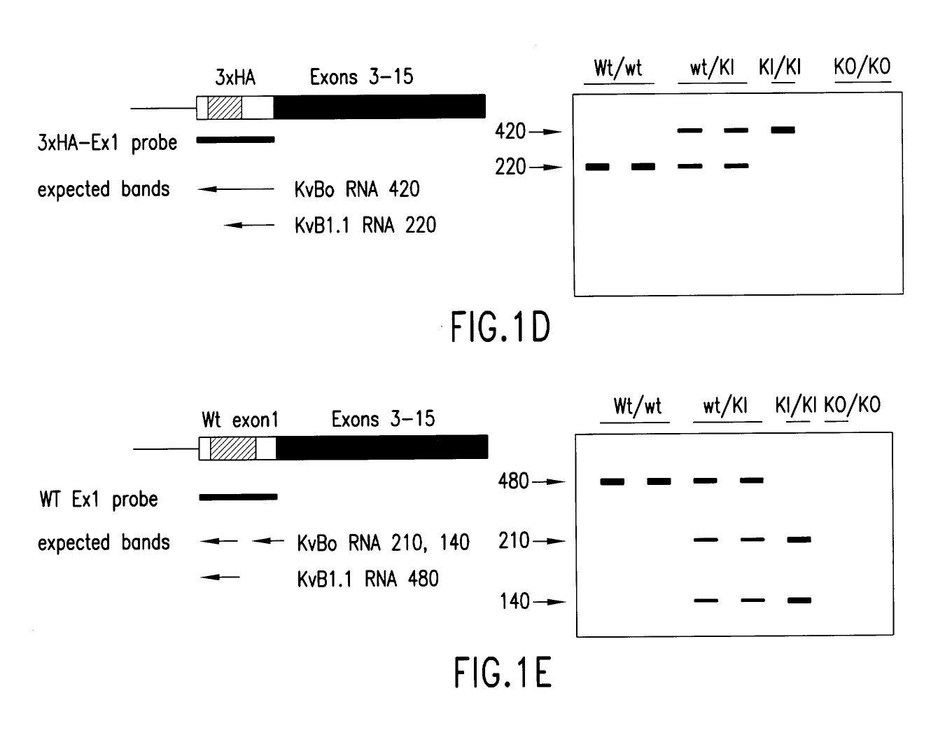

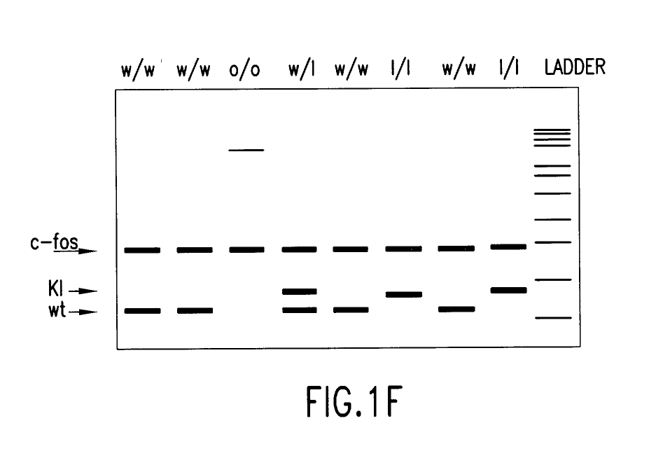

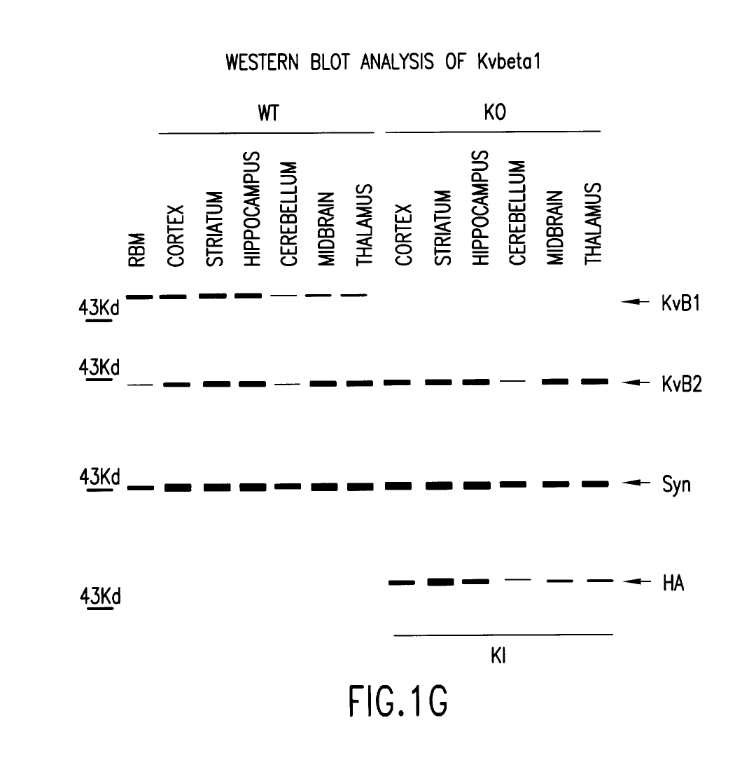

The knock-in transgenes of the present invention preferably include foreign DNA sequences which inactivate only a portion of the Kvβ1.1 gene, in particular the “ball” domain in the amino-terminus which enables rapid inactivation of the K⁺ channel. The mutation may be a replacement mutation, an insertion mutation, a frameshift mutation, or a stop codon mutation which is substituted or inserted into the endogenous Kvβ1.1 gene. Such transgenes preferably contain at least one DNA sequence that is identical to some portion of the endogenous Kvβ1.1 gene to be functionally disrupted. The presence of the mutation in all or a portion of codons 1-70 (SEQ ID NO: 1) of a Kvβ1.1 allele functionally disrupts the expression of the inactivation domain (see e.g., FIG. 1A).

Preferably, the mutation results in functional disruption either by interference in initiation of transcription or translation, or by premature termination of transcription or translation of the inactivation domain of the Kvβ1.1 protein. More preferably, the transgenes are replacement-type mutations because they increase the stability of the construct in the endogenous Kvβ1.1 gene and reduce the likelihood of secondary recombination and reversion.

In a preferred embodiment, the nucleic acid constructs (the “targeting constructs”) used to generate the transgenes are produced by ligation of an expression cassette encoding an immunoreactive epitope tag and/or a selectable marker into the DNA sequence encoding the Kvβ1.1 gene products. The targeting constructs further comprise at least one sequence portion flanking the expression cassette which is homologous to at least a part of the endogenous Kvβ1.1 gene. The presence of one homologous sequence adjacent to the amino-terminus will enable an insertion mutation in the inactivation domain of the Kvβ1.1 gene. In a most preferred embodiment however, the targeting construct comprises both an immunoreactive epitope tag and a selectable marker which are both in turn flanked by a pair of sequences homologous to endogenous Kvβ1.1 (see e.g., Example 1). The pair of sequences are preferably homologous to endogenous Kvβ1.1 sequences which encode codons 1-70, the inactivation domain required for channel inactivation.

Alternatively, the cassette is also inserted in a location such that splicing out of the cassette introduces a frameshift mutation resulting in non-functional reversions. In a further alternative embodiment, the cassette may provide a stop codon to prematurely terminate transcription or translation. While it is possible to use these alternative embodiments to develop the transgenic mammals of the present invention, detection of successful integration into the endogenous sequences by these methods requires extensive use of Southern hybridization and PCR analysis. It is therefore preferable to incorporate an immunoreactive epitope tag which allows for ease in tracking and isolating the knock-in Kvβ1 subunit.

Immunoreactive epitope tags may be fused into the targeting construct to provide an epitope to which an anti-tag antibody can selectively bind. The epitope tag is preferably placed in the amino-terminus of the targeting construct, corresponding to codons 1-70 of the Kvβ1.1 gene, and more preferably to codons 1-36. After transcription and translation, the presence of such epitope-tagged forms in a knock-in Kvβ1 subunit can be detected using an antibody against the tag subunit. Also, provision of the epitope tag enables the Kvβ1 subunit to be readily purified by affinity purification using an anti-tag antibody or another type of affinity matrix that binds to the epitope tag. Alternatively, the targeting construct may be fused with a nucleic acid sequence encoding an immunoglobulin or a particular region of an immunoglobulin, such as the Fc region of an IgG molecule, to allow specific binding to an extraneous epitope tag.

Various epitope tags and their respective antibodies are well known in the art. Examples include poly-histidine (poly-his) or poly-histidine-glycine (poly-his-gly) tags; the flu HA tag polypeptide and its antibody 12CA5; the c-myc tag and the 8F9, 3C7, 6E10, G4, B7 and 9E10 antibodies thereto; and the Herpes Simplex virus glycoprotein D (gD) tag and its antibody. Other epitope tags include the Flag-peptide; the KT3 epitope peptide; tubulin epitope peptide; and the T7 gene 10 protein peptide tag.

Preferred “positive” selectable markers include the bacterial aminoglycoside 3′ phosphotransferase gene (‘neo’) which confers resistance to the drug G418 in mammalian cells, the bacterial hygromycin G phosphotransferase gene (hyg) which confers resistance to the antibiotic hygromycin, the bacterial xanthine-guanine phosphoribosyl transferase gene (also referred to as the gpt gene) which confers the ability to grow in the presence of mycophenolic acid, as well as the hprt gene, the nDtll gene, or other genes which confer resistance to amino acid or nucleoside analogues; or antibiotics, etc. The DNA encoding the positive selectable marker in the transgene (e.g. neo^{R}) is generally linked to an expression regulation sequence that allows for its independent transcription in ES cells. Moreover, “negative” selectable markers may also be used, which encode an enzymatic activity whose expression is cytotoxic to the cell when grown in an appropriate selective medium. For example, the HSV-tk gene is used as a negative selectable marker. Expression of the HSV-tk gene in cells grown in the presence of gancyclovir or acyclovir is cytotoxic; thus growth of cells in selective medium containing gancyclovir or acyclovir selects against cells capable of expressing a functional HSV-TK enzyme. In an example using both positive and negative selectable markers, cells which express an active HPRT enzyme are unable to grow in the presence of certain nucleoside analogues (such as 6-thioguanine, 8-azapurine, etc.), but are able to grow in media supplemented with HAT (hypoxanthine, aminopterin, and thymidine). Conversely, cells which fail to express an active HPRT enzyme are unable to grow in media containing HATG, but are resistant to analogues such as 6-thioguanine, etc.

B. Homologous Recombination

Preferably, the present invention utilizes homologous recombination to control the site of integration of a specific DNA sequence (transgene) into the naturally-occurring Kvβ1.1 sequence of a mammalian cell and thereby disrupt normal functionality of that gene. Homologous recombination is well-known in the art. In summary, homologous recombination is a natural process that occurs during mitosis whereby two nucleic acid molecules having identical or substantially similar (i.e. “homologous”) sequences essentially “switch” the DNA sequences adjacent to or in-between the homologous sequences, such that one region of each initially present molecule is now ligated to a region of the other molecule.

The process of homologous recombination can be manipulated to target specific genes by methods well known to those skilled in the art. Most techniques utilize selectable markers, either positive or negative or both, to identify and isolate transformed cells. Moreover, it is possible to increase the frequency of recombination between DNA molecules by using stimulatory agents such as trimethylpsoralen or UV light.

In a preferred embodiment, the targeting construct comprises an expression cassette, which in turn comprises an immunoreactive epitope tag and/or a selectable marker, and the expression cassette is flanked by at least one sequence which is homologous to a portion of the endogenous Kvβ1.1 gene. Preferably, the targeting construct is designed such that an immunoreactive epitope tag is ligated into a nucleic acid sequence corresponding to all or a portion of the targeted region (i.e. the inactivation domain encoding codons 1-70, particularly 1-36) of the endogenous Kvβ1 gene. The immunoreactive epitope tag permits for subsequent identification and isolation by Western blot or immunoassay. Alternatively, or in addition to the immunoreactive epitope tag, a positive selectable marker such as neo may be inserted into or near a sequence corresponding to the inactivation domain of endogenous Kvβ1 such that expression of the cassette provides antibiotic resistance (neo provides resistance to the antibiotic G418). In the most preferred embodiment, the expression cassette comprises both an immunoreactive epitope tag and a selectable marker. Moreover, the targeting construct further comprises a pair of first and second sequences which flank both sides of the expression cassette and are homologous to portions of endogenous Kvβ1.1 at its amino-terminus.

These first and second homologous sequences of the endogenous gene target the transgene to the specific Kvβ1 allele in the ES cell. In a successful example of targeting by homologous recombination, the nucleic acid region lying between the homologous sequences is specifically integrated into, and replaces a portion of, the targeted endogenous gene in one allele of the ES cell. Consequently, one copy of the targeted Kvβ1 allele is disrupted by homologous recombination with the transgene. Selection with G418 thereafter selects for transfected ES cells containing the transgene integrated into the genome by homologous recombination. Moreover, detection of the transgene as incorporated into the encoded Kvβ1 subunit may be performed by affinity binding with an antibody to the immunoreactive epitope tag.

In the most preferred embodiment of the invention, it is not desirable to have an expressed antibiotic resistance gene incorporated into the cells of a transgenic animal, i.e. the antibiotic resistance gene may have deleterious effects, “neighborhood effects” on the neighboring genes, or on the targeted gene, even at long distances. Therefore, in a most preferred embodiment, one or more genetic elements are included in the knock-in construct that permit the antibiotic resistance gene to be excised once the construct has undergone homologous recombination with the endogenous Kvβ1.1 gene.

In this most preferred embodiment, the antibiotic resistant selectable marker is flanked by repeat recombination sites, such as LoxP sites. The presence of direct repeats such as loxP in genomic DNA enables a recombinase protein such as Cre-recombinase to excise the intervening DNA (the neo gene), leaving only a single LoxP site in the targeted locus. The presence of this LoxP site rarely, if ever, affects expression. In this embodiment, the selectable marker is inserted into an intron and is only used to identify the successful integration of targeting construct into the endogenous gene. Once transfected, Cre-recombinase can be expressed in ES cells or in specific tissues of transgenic mice to efficiently remove the neo marker. Westphal et al. Proc Natl Acad Sci (1996) 93:5860.

As mentioned above, mutation of the Kvβ1 inactivation domain may be performed by generating a mutation in a portion of exon 1.1, as illustrated in Example 1, most preferably in codons 1-36, and in particular 1-15. It has further been noted that conversion of the cysteine residue at codon 7 to any other codon except a serine residue (missense, stop codon, etc.), will disrupt the inactivation domain. The mutation may be performed by replacing part of, or inserting into, the endogenous exon 1.1 sequence either an immunoreactive epitope tag, a selectable marker, a stop codon or alternatively, by causing a frameshift mutation. In the most preferred embodiment however, only the immunoreactive epitope tag disrupts the amino-terminus and remains in the final targeted gene. The selectable marker is excised after transformation. Preferably, the epitope tag is integrated into the inactivation domains of the Kvβ1.1 gene in both alleles of the cell, but the frequency of such an event occurring is low (the square of the frequency of a single mutational event). Therefore, cross-breeding of heterozygous animals may be performed to produce homozygous animals with the knock-in Kvβ1.1 in both alleles.

Preferably, the DNA molecules are double stranded, but single stranded DNA molecules may also be used in the invention. In addition, the DNA molecules may be introduced to the cell as either DNA or RNA, which may in turn be converted to DNA by reverse transcriptase or by other means. An illustrative best mode for conducting the invention is provided in Example 1.

C. Transformation of Cells.

To produce the knock-in mammals of the invention, cells are transformed by introducing the targeting construct described above into totipotent cells, such as embryonal stem (“ES”) cells, which are capable of giving rise to all cell types of an embryo, including germ cells. A number of ES cells may be used in the present invention. ES cells from mice have been isolated by culturing cells derived from murine blastocysts (Evans et al. (1981) Nature 292:154-156; Bradley et al. (1984) Nature 309:255-258; Gossler et al. (1986) Proc. Acad. Sci USA 83:9065-9069; and Robertson et al. (1986) Nature 322:445-448). Preferably, however, primary isolates of ES cells are used. Primary isolates may be obtained directly from embryos such as the CCE cell line, or from clonal isolation of ES cells therefrom (Schwartzberg et al. (1989) Science 212:799-803). It is generally understood that primary isolates are more efficient for differentiating into a mammal, and in particular, clonally-selected ES cells are approximately 10-fold more effective in producing transgenic mammals than the CCE progenitor cell line. Some examples of clonally-isolated ES cell lines include AB1 (hprt⁺) and AB2.1 (hprt⁻).

Preferably, the ES cells are cultured on stromal cells such as primary embryonic G418 R fibroblast cells and/or STO cells. Fibroblast and/or stromal cells prevent clonal overgrowth of abnormal ES cells. Most preferably, the cells are cultured in the presence of a differentiation-inhibiting factor (“DIF”), such as leukocyte inhibiting factor (“LIF”), to prevent premature differentiation. Other known DIF's include Oncostatin M, interleukin 6 (IL-6) with soluble IL-6 receptor (sIL-6R) and ciliary neurotrophic factor (CNTF), T-LIF (U.S. Pat. No. 5,849,991) and certain cytokines. Furthermore, it is possible to transform stromal cells, with an expressible DIF, upon which ES cells may then be cultured.

The methods of introducing exogenous nucleic acid into mammalian hosts and host cells are well known in the art, and vary depending on the host cell. Techniques include electroporation, DEAE-dextran-mediated transfection, calcium phosphate co-precipitation, protoplast or spheraplast fusion, lipofection, micro-injection or viral infection. The transfected ES cells can then be introduced into a blastocoel in a blastocyst stage embryo and contribute to the germ line of the transgenic mammal.

In addition, prior to introduction of the ES cells into the blastocoel, various selection protocols (e.g., neo selectable marker) as described above may be used to select for transfected ES cells which have incorporated the transgene. Alternatively, southern hybridization or PCR can be used to determine integration of the transgene.

1. Microinjection

In addition, alternative methods are known in the art for the generation of transgenic mammals containing the transgene. Embryonal cells at various stages of development can be used, according to correspondingly different techniques. Where the zygote is used, micro-injection is the preferred technique as described in U.S. Pat. No. 4,873,191, the contents of which are herein incorporated by reference. In the mouse, injection of 1-2 picoliters (pl) of DNA solution can be made when the male pronucleus reaches a diameter of approximately 20 micrometers. Furthermore, it is possible to inject the zygote prior to first cleavage, thereby ensuring incorporation of the construct into all somatic and germ cells of the transgenic animal (Brinstei, et al. (1985) Proc. Natl. Acad. Sci. USA 82, 4438-4442). The resulting transgenic mammal will be capable of transmitting the foreign DNA to future offspring. Moreover, in this embodiment it is not necessary to first introduce the targeting construct into a self-replicating plasmid or virus.

2. Retroviral Transformation

In another embodiment, retroviral infection is used to introduce a transgene into a non-human mammal. The technique of retroviral infection uses embryos which have been cultured in vitro to the blastocyst stage, and targets the blastomeres for infection (Jaenich (1976) Proc. Natl. Acad. Sci USA 73:1260-1264). Enzymatic treatment removes the zona pellucida of the blastocysts and facilitates infection via a replication-defective retrovirus carrying the transgene (Van der Putten, et al. (1985) Proc. Natl. Acad. Sci. USA 82, 6148-6152). The transfected blastomeres are then cultured on a monolayer of virus-producing cells. In addition, virus or virus-producing cells can be injected into the blastocoel (Jahner et al. (1982) Nature, 298:623-628). In this embodiment, the resulting transgenic mammals will be mosaic for the transgene, since only a subset of the cells will have incorporated the transgene. In addition, retroviral insertion of the transgene may occur at different positions in the genome which generally will segregate in the offspring. In slight variation of this technique, it is also possible to introduce the transgenes into the germline via intrauterine retroviral infection of the midgestation embryo and thereby generate more comprehensive integration of the transgene (Jahner et al. (1982) supra).

3. Electroporation into ES Cells

In a most preferred embodiment, the transgene containing the targeting construct is introduced to the ES cell by electroporation (Toneguzzo et al., (1988) Nucleic Acids Res., 16:5515-5532; Quillet et al. (1988) J. Immunol., 141:17-20; Machy et al. (1988) Proc. Natl. Acad. Sci. USA, 85:8027-8031). The cells are then cultured and selected for cells which have successfully integrated the transgene, as described above (e.g., neo in G418 medium). Alternatively, the transgene may be detected by radiolabelled nucleotides, or by other assays of detection which do not require the expression of the selectable marker sequence, such as by PCR amplification techniques.

4. Other Non-Human Transgenic Mammals

One of skill in the art will recognize that there are a number of other natural or transgenic mammals, in addition to mice, which may be used in the invention. As with the murine model, the zygotes or ES cells of these animals may be used as embryonic targets for introducing the transgenes. In each instance, a transgenic non-human mammal is formed having the desirable defective Kvβ subunit phenotype that is characteristic to the mammal.

While the development of transgenic mammals by micro-injection has been greatest in mice, it is possible to generate other transgenic mammals by micro-injection of zygotes as well, including rabbits, sheep, cattle, and pigs (Jaenisch (1988) Science 240:1468-1474; Hammer et al. (1986) J. Anima1. Sci, 63:269; Hammer et al. (1985) Nature 315:680; Wagner et al. (1984) Theriogenology 21:29). Most preferably, however, the transgenic mammal of the present invention is a mouse or a rat, which has a micro-injection success rate of approximately 10-30%. In addition, retroviral-mediated methods or electroporation techniques using other non-human mammalian ES cells may be used. The derivation of ES cell lines for mice and pigs have previously been reported in the art (Robertson, Embryo-Derived Stem Cell Lines, In: Teratocarcinomas and Embryonic Stem Cells: A Practical Approach (E. J. Robertson, ed.), IRL Press, Oxford (1987); PCT Publication No. WO/90/03432; PCT Publication No. 94/26884). In addition, ES cell lines may be derived or isolated from any species (for example, chicken, etc.), although cells derived or isolated from mammals such as rodents, rabbits, sheep, goats, fish, pigs, cattle, primates and humans are preferred. In the most preferred embodiment of the invention, murine ES cells are used.

As is well-appreciated in the art, transformation of the Kvβ1.1 allele of other non-human mammals requires the Kvβ1.1 gene sequence for that species. Murine Kvβ1.1 gene sequence is on deposit at Genbank as Accession No. AF033003; while rat Kvβ1.1 sequence is on deposit at Genbank as Accession No. X70662. The structure and function of the Kvβ1.1 gene in other non-human mammals is also well-known in the art and is publicly available. Moreover, the desired Kvβ1.1 sequence for a given species can be readily obtained by using probes from known Kvβ1.1 sequences, by hybridization or other such techniques well-known in the art. The genome library of the target mammal may be screened (i.e., a Southern Blot) using low stringency with appropriate probes, and the remaining portions of the gene sequenced by routine methods.

Once the target Kvβ1.1 sequence for the desired species is obtained, a target construct can be designed as described above (using various replacement mutations, insertion mutations, stop codon mutations or frame-shift mutations), to cause inactivation of the ball domain of Kvβ1.1. Then, using methods as described above and in Example 1 below, one skilled in the art may proceed to introduce the targeting vector to an ES cell or zygote of the species and thereby generate a transgenic mammal of the invention.

II. In vitro Binding Assays

A. Pre-Screening Assays

In one embodiment of the present invention, the knock-in Kvβ1.1 subunit (incorporating the replacement, insertion, stop codon or frame-shift mutation of the inactivation domain) is useful for pre-screening test compounds for the ability to modulate activity of a Kvβ1.1 subunit. In these embodiments, the knock-in Kvβ1.1 subunit is particularly useful for identifying compounds that interfere with a specified functionality of the normal Kvβ1.1 subunit, ie., the ability of the Kvβ1.1 subunit to either co-associate with Kv1 family α subunits or its ability to inactivate the potassium channel. In one pre-screening embodiment, the test compounds are contacted with a knock-in Kvβ1.1 subunit and a test compound is selected which is capable of providing a detectable change in the activity of the knock-in Kvβ1.1 subunit. Most likely, the detectable change will be detection of binding of the test compound to the knock-in Kvβ1.1 subunit. In addition, an immunoassay may be used to determine whether the knock-in Kvβ1.1 subunit co-associates with a Kv1 family αsubunit, or whether one of the test compounds prevents co-association. In this embodiment, the binding assays can be used to pre-screen for test compounds which have a preference in binding to Kvβ1.1 over Kv1 family α subunits, or vice versa.

Alternatively, using the binding assays in a comparative approach, test compounds are pre-screened for their ability to bind to a wild-type (“WT”) Kvβ1.1 subunit but not to a mutated knock-in (“KI”) Kvβ1.1 subunit, thereby identifying potential candidates for inhibiting channel inactivation. Each of these pre-screening binding assays may be used prior to performing the additional binding assays and complex functional assays described below.

Binding assays using the target binding protein (KI, WT, KO Kvβ1.1 and Kv1 α) immobilized or not, are well known in the art and may be used for screening test compounds. Purified cell-based or protein based (cell free) screening assays may be used to identify such compounds. For example, a mutated Kvβ1.1 (KI) subunit may be immobilized in purified form on a carrier and binding to the mutated Kvβ1.1 subunit may be measured in the presence and absence of potential inhibiting compounds, competitive binding may be measured in the presence of Kv1 family α subunits. Conversely, the Kv1 family α subunits may be immobilized on a carrier and subjected to test compounds in the presence of a mutated KI Kvβ1.1.

The in vitro binding assays may be manipulated for a variety of useful comparative analyses to reveal additional modulators of Kvβ1.1 subunit activity. In one preferred comparative approach, a purified wild-type Kvβ1.1 subunit (“WT”) may be immobilized on the carrier and binding to the WT Kvβ1.1 may be measured in the presence and in the absence of test compounds, and then compared to a knock-in Kvβ1.1 which is also immobilized in purified form on a carrier and subjected to contact with test compounds. Both WT and KI Kvβ1.1 subunits may be present in the same test sample, or alternatively, may be in separate test samples. This embodiment may be useful for identifying compounds capable of mimicking the activity of a KI Kvβ1.1 subunit. Test compounds which bind successfully to the WT but not the KI may then be subjected to additional binding assays and functional assays as described below, for use as a potential therapeutic agent for anxiety disorders. The KO subunit may further be used as a control against the WT and KI genotypes. A suitable binding assay may alternately employ purified polypeptide forms of WT, KI or KO Kvβ1.1 subunits, or may employ cells characterized by expressing each of the above genotypes.

Most preferably, the pre-screening assays are designed such that a large volume of test compounds may be simultaneously screened and evaluated against each other for binding activity with the subject WT, KI or KO Kvβ1 subunits.

B. High-Throughput Screening

Preferably, the pre-screening assays of this invention are amenable to “high-throughput” modalities. Traditional research methodologies entailed the study of a “lead compound” having some desirable property (modulatory, inhibitory, etc.) then modifying the lead compound to create variants and evaluating the efficacies of the variants. High-throughput screening on the other hand, enables for more rapid drug discovery and has become the preferred method for generating test compounds.

In one preferred embodiment, high throughput screening methods involve providing a library containing a large number of test compounds potentially having the desired activity. Such “combinatorial chemical libraries” are then screened in one or more assays, as described herein, to identify those library members (a particular chemical species or subclasses) that display a desired characteristic activity. The compounds which are identified can then serve as conventional “lead compounds” or can themselves be used as potential or actual therapeutics.

1. Combinatorial Chemical Libraries for Potential Kvβ1.1 Modulators

A combinatorial chemical library is a collection of diverse chemical compounds generated by either chemical synthesis or biological synthesis by combining a number of chemical “building blocks”, such as reagents. For example, a linear combinatorial chemical library such as a polypeptide library is formed by combining a set of chemical building blocks called amino acids in every possible way for a given compound length (i.e., the number of amino acids in a polypeptide compound). Millions of chemical compounds can be synthesized through such combinatorial mixing of chemical building blocks. Preferably, libraries are screened for candidate small molecules. Examples of such libraries include spatially addressable parallel solid phase or solution phase libraries or synthetic libraries made from deconvolution, “one-bead one-compound” methods or by affinity chromatography selection.

Preparation and screening of combinatorial chemical libraries is well known. Examples include, but are not limited to, peptide libraries (see, e.g., U.S. Pat. No. 5,010,175). Peptide synthesis is by no means the only approach envisioned and intended for use with the present invention. Other chemistries for generating chemical diversity libraries can also be used. Such chemistries include, but are not limited to: peptides (PCT Publication WO 91/19735), encoded peptides (PCT Publication WO 93/20242), random bio-oligomers (PCT Publication WO 92/00091), benzodiazepines (U.S. Pat. No. 5,288,514), diversomers such as hydantoins, benzodiazepines and dipeptides (Hobbs et al. (1993) Proc. Nat. Acad. Sci. USA 90:6909-6913), vinylogous polypeptides (Hagihara et al. (1992) J. Amer. Chem. Soc. 114: 6568), nonpeptidal peptidomimetics with a Beta- D- Glucose scaffolding (Hirschmann et al. (1992) J. Amer. Chem. Soc. 114:9217-9218), analogous organic syntheses of small compound libraries (Chen et al. (1994) J. Amer. Chem. Soc. 116:2661), oligocarbamates (Cho et al. (1993) Science 261:1303), and/or peptidyl phosphonates (Campbell et al. (1994) J. Org. Chem. 59:658). See, generally, Gordon et al. (1994) J. Med. Chem. 37:1385, nucleic acid libraries (see, e.g., Strategene Corp.), peptide nucleic acid libraries (see, e.g., U.S. Pat. No. 5,539,083) antibody libraries (see, e.g., Vaughn et al. (1996) Nature Biotechnology, 14(3):309-314, and PCT/US96/10287), carbohydrate libraries (see, e.g., U.S. Pat. No. 5,593,853), and small organic molecule libraries (see, e.g., benzodiazepines, Baum (1993) C&EN, January 18, page 33; isoprenoids, U.S. Pat. No. 5,569,588; thiazolidinones and metathiazanones, U.S. Pat. No. 5,549,974, pyrrolidines, U.S. Pat. Nos. 5,525,735 and 5,519,134; morpholino compounds, U.S. Pat. No. 5,506,337; benzodiazepines, U.S. Pat. No. 5,288,514; and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.).

A number of well known robotic systems have also been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.) which mimic the manual synthetic operations performed by a chemist. Any of the above devices are suitable for use with the present invention. The nature and implementation of modifications to these devices (if any) so that they can operate as discussed herein will be apparent to persons skilled in the relevant art.

In addition, numerous combinatorial libraries are themselves commercially available (see, e.g., ComGenex, Princeton, N.J.; Asinex, Moscow, RU; Tripos, Inc., St. Louis, Mo.; ChemStar, Ltd., Moscow, RU; 3D Pharmaceuticals, Exton, Pa.; Martek Biosciences, Columbia, Md., etc.).

2. High-Throughput Assays of Chemical Libraries

As mentioned above, the pre-screening assays for test compounds that modulate the binding specificity and/or activity of Kvβ1.1 polypeptides are preferably amenable to high-throughput screening. More preferably, the pre-screening assays are capable of detecting inhibition of the characteristic activity of the Kvβ1.1 polypeptide. High throughput assays for the presence, absence, or quantification of particular nucleic acids or protein products are well known to those of skill in the art.

Binding assays are similarly well known. Thus, for example, U.S. Pat. No. 5,559,410 discloses high throughput screening methods for proteins, U.S. Pat. No. 5,585,639 discloses high throughput screening methods for nucleic acid binding (i.e., in arrays), while U.S. Pat. Nos. 5,576,220 and 5,541,061 disclose high throughput methods of screening for ligand/antibody binding.

The high throughput screening systems for use in the pre-screening assays are all commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols for each high throughput system. Thus, for example, Zymark Corp. provides technical bulletins describing screening systems for detecting the modulation of gene transcription, ligand binding, and the like.

C. Assays for Assessing Efficacy of a Test Compound

Once a number of test compounds have been identified, in vitro binding assays and functional assays may be used to further assess the efficacies of such test compounds for modulating the activity of Kvβ1.1.

In vitro binding assays may be used to quantify and measure the binding capabilities of the test compound for comparative evaluation. In an embodiment directed to assessing the test compound's effect on Kvβ1.1 subunit co-association, a first binding mixture is formed by combining a knock-in Kvβ1.1 subunit, or fragments thereof, and a Kv1 α subunit, and then the amount of binding in the first binding mixture is measured. A second binding mixture is also formed by combining knock-in Kvβ1.1 subunit, a Kv1 α subunit and the test compound and then the amount of binding in the second binding mixture is measured. The amounts of binding in the first and second binding mixtures are compared. A test compound is considered to be capable of preventing co-association of Kvβ1.1 with Kv1 α subunits if there is a decrease in binding with the second binding mixture as compared to the first binding mixture. More preferably, the assay will quantify the degree to which the test compound reduces the binding activity of a knock-in Kvβ1 subunit or its fragments, preferably by greater than 10%, more preferably greater than about 50% or more. Optionally, additional agents may be added to study their interaction with the β1 and Kv1 α subunits, i.e., β2 subunits. The formulation and optimization of binding mixtures is within the level of skill in the art. Such binding mixtures may also contain buffers and salts necessary to enhance or optimize binding, and additional control assays may be included in the screening assay of the invention.

On the other hand, one may assess the efficacy of a test compound for inhibiting inactivation of potassium channels by contacting the test compound with a wild-type Kvβ1.1 subunit and a knock-in Kvβ1.1 subunit, and detecting a change in the wild-type Kvβ1.1 subunit but no change in the activity of the knock-in Kvβ1.1 subunit. In this latter embodiment, an effective test compound will reduce WT Kvβ1.1 activity by preferably greater than 10%, more preferably greater than about 50% (whereas KI activity and KO activity are not reduced). In this latter embodiment, wild type Kvβ1.1 activity is measured as a function of the subunit's binding capabilities, whereas other functional assays described below may be used to specifically assess the test compound's efficacy on inhibiting channel inactivation. By these means, test compounds having inhibitory activity for Kvβ1 subunits which may be suitable as therapeutic agents can be identified. The wild-type and knock-in Kvβ1.1 subunits may be combined in one test sample, or may alternately be provided such that the wild-type Kvβ1.1 is in a first test sample and the knock-in Kvβ1.1 is in a second test sample. Furthermore, the test samples may comprise cells, tissues or transgenic mammals.

Binding assays, such as Western blots and immunoassays, may be used to determine the amount of Kvβ1 polypeptide present. Standard analytic methods for detection and/or quantification of Kvβ1 subunits include electrophoresis, capillary electrophoresis, high performance liquid chromatography (HPLC), thin layer chromatography (TLC), hyperdiffusion chromatography, and the like, or various immunological methods such as fluid or gel precipitation reactions, immunodiffusion (single or double), immunoelectrophoresis, radioimmunoassay (RIA), enzyme-linked immunosorbent assays (ELISAs), immunofluorescent assays, western blotting, and the like. In one preferred embodiment, the subject Kvβ1.1 polypeptide(s) is detected/quantified in an electrophoretic protein separation (e.g., a 1- or 2-dimensional electrophoresis), which is well-known in the art. Where Western blot (immunoblot) analysis is used to detect and quantify the presence of KVβ1 polypeptide(s), the polypeptide(s) in the test sample is separated by gel electrophoresis on the basis of molecular weight, transferred to an appropriate support, (e.g., nitrocellulose or nylon filter), and incubated in the sample with the antibodies specific to the target polypeptide(s). As is well-appreciated in the art, the target polypeptides (β1.1 or Kv1 α subunits) may be directly labeled themselves, or may subsequently be detected using labeled antibodies (e.g., labeled sheep anti-mouse antibodies).

In a preferred embodiment, immunoassays are used to detect the presence of the Kvβ1 subunit. As used herein, an immunoassay is an assay that utilizes an antibody to specifically bind to the analyte (e.g., the target polypeptide(s)). The immunoassay is thus characterized by detection of specific binding of a polypeptide of this invention to an antibody as opposed to the use of other physical or chemical properties to isolate, target, and quantify the subject Kvβ1 analyte. Any of a number of well recognized immunological binding assays (see, e.g., U.S. Pat. Nos. 4,366,241; 4,376,110; 4,517,288; and 4,837,168) are well suited to detection or quantification of the polypeptide(s) identified herein. Immunological binding assays (or immunoassays) typically utilize a “capture agent” to specifically bind to and often immobilize the analyte (the subject WT, KI or KO Kvβ1.1 polypeptide). In preferred embodiments, the capture agent is an antibody. In a most preferred embodiment, immunoassays are used to determine whether the subject Kvβ1.1 subunit (WT, KI or KO) is co-associating with the Kv1 αsubunits, as described in Example 1 below.

Generally, the target subunits (WT, KI, KO or Kv1 α-) may be immobilized on an insoluble support having isolated sample receiving areas (e.g., a microtiter plate, an array, beads, membranes, etc.). Alternatively, cells comprising the Kvβ1 proteins can be used in the assays. The insoluble supports may be made of any material to which the target subunits can be bound and readily separated from soluble material, and which are otherwise compatible with the overall method of screening. The surface of such supports may be solid, porous or of any convenient shape and are typically made of glass, plastic (e.g., polystyrene), polysaccharides, nylon or nitrocellulose, Teflon®, etc. Microtiter plates and arrays are especially convenient because a large number of assays can be carried out simultaneously using small amounts of reagents and samples. The particular manner of binding the target subunit is not crucial so long as it is compatible with the reagents and overall methods of the invention, maintains the activity of the target subunit and is nondiffusable. Preferred methods of binding include the use of antibodies (which do not sterically block either the ligand binding site or activation sequence when the protein is bound to the support), direct binding to “stick” or ionic supports, chemical crosslinking, synthesis of the protein or agent on the surface, etc. Following binding of the protein or agent, excess unbound materials are removed by washing. The sample receiving areas may then be blocked through incubation with bovine serum albumin (BSA), casein or other innocuous protein or other moiety.

As mentioned above, in a preferred embodiment, a target Kvβ1 (WT, KI or KO) protein is bound to the support, and the test compound is added to the assay. Alternatively, the test compound may be bound to the support and the Kvβ1 protein added. Novel test compounds of potential interest as therapeutic agents include specific antibodies and non-natural binding agents identified in screens of chemical libraries, peptide analogs, etc. Of particular interest are screening assays for agents that have a low toxicity for human cells. A wide variety of assays may be used to identify and assess the efficacies of test compounds, including labeled in vitro protein-protein binding assays, electrophoretic mobility shift assays, immunoassays for protein binding, phosphorylation assays and the like.

The determination of the binding of the test compound to the Kvβ1 protein may be done in a number of ways. In a preferred embodiment, the test compound is labeled, and binding determined directly. For example, this may be done by attaching all or a portion of the Kvβ1 protein to a solid support, adding a labeled candidate agent (for example, a fluorescent label), washing off excess reagent, and determining whether the label is present on the solid support. Various blocking and washing steps may be utilized as is known in the art.

By “labeled” it is herein meant that the compound is either directly or indirectly labeled with a molecule or compound which provides a detectable signal, e.g. radioisotope, fluorescers, enzyme, antibodies, particles such as magnetic particles, chemiluminescers, or specific binding molecules, etc. Specific binding molecules include pairs, such as biotin and streptavidin, digoxin and antidigoxin, etc. For the specific binding members, the complementary member would normally be labeled with a molecule which provides for detection, in accordance with known procedures, as outlined above. The label can directly or indirectly provide a detectable signal. In a most preferred embodiment, the label is the immunoreactive epitope tag, as previously discussed.

In some embodiments, only one of the components is labeled. For example, the proteins (or proteinaceous candidate agents) may be labeled at tyrosine positions using ¹²⁵I, or with fluorophores. Alternatively, more than one component may be labeled with different labels for example, using ¹²⁵I for the proteins and a fluorophor for the candidate agents.

In a preferred embodiment, the binding of the test compound is determined through the use of competitive binding assays. In this embodiment, the competitor is a binding moiety known to bind to the target Kvβ1 subunit, i.e. the competitor is an Kvα1.1 subunit. In one embodiment, the test compound is labeled. Either the test compound, or the competitor, or both, is added first to the protein for a time sufficient to allow binding. Incubation may be performed at any temperature which facilitates optimal activity, typically between 4 and 37° C. for binding of β1 and β1.1. Incubation periods are selected for optimum activity, but may also be optimized to facilitate rapid high throughput screening. Typically between 10 minutes and 1 hour will be sufficient. Excess reagent is generally removed or washed away. The second component is then added, and the presence or absence of the labeled component is followed, to indicate binding. In a preferred embodiment, the competitor (i.e., Kvα1.1) is added first, followed by the test compound. Displacement of the competitor is an indication that the test compound is binding to the subject Kvβ1 protein and thus capable of binding to, and potentially modulating, the activity of the subject Kvβ1 protein. In this embodiment, either component can be labeled. Thus, for example, if the competitor is labeled, the presence of label in the wash solution indicates displacement by the agent. Alternatively, if the test compound is labeled, the presence of the label on the support indicates displacement.

In an alternative embodiment, the test compound is added first, with incubation and washing, followed by the competitor (i.e., Kvα1.1 subunit). The absence of binding by the competitor may indicate that the test compound is bound to the Kvβ1 protein with a higher affinity. Thus, if the test compound is labeled, the presence of the label on the support, coupled with a lack of competitor binding, may indicate that the test compound is capable of binding to the Kvβ1 protein.

In one preferred embodiment, the methods comprise differential screening to assess test compounds that are capable of modulating the activity of the subject Kvβ1 subunits. In this embodiment, the methods comprise combining a Kvβ1 protein and a competitor in a first sample. A second sample comprises a test compound, a Kvβ1 protein and a competitor. The binding of the competitor is determined for both samples, and a change, or difference in binding between the two samples indicates the presence of a test compound capable of binding to the Kvβ1 protein and potentially modulating its activity. That is, if the binding of the competitor is different in the second sample relative to the first sample, the test compound is capable of binding to the Kvβ1 protein.

Alternatively, a preferred embodiment utilizes differential screening to assess the efficacies of test compounds that bind to the WT Kvβ1 protein, but cannot bind to KI or KO Kvβ1 subunits, or which can bind to KI but not KO. The structure of the Kvβ1 protein may be modeled, and used in rational drug design to synthesize agents that interact with that site. Test compounds that affect Kvβ1 activity are also identified by screening compounds for the ability to either enhance or reduce the activity of the protein.

Positive controls and negative controls may be used in the assays, such as a completely non-functional Kvβ1.1 subunit (KO). Preferably all control and test samples are performed in at least triplicate to obtain statistically significant results. Incubation of all samples is for a time sufficient for the binding of the agent to the protein. Following incubation, all samples are washed free of non-specifically bound material and the amount of bound, generally labeled, test compound is determined. For example, where a radiolabel is employed, the samples may be counted in a scintillation counter to determine the amount of bound compound.

A variety of other reagents may be included in the screening assays. These include reagents like salts, neutral proteins, e.g., albumin, detergents, etc., which may be used to facilitate optimal protein-protein binding and/or reduce non-specific or background interactions. Also reagents that otherwise improve the efficiency of the assay, such as protease inhibitors, nuclease inhibitors, anti-microbial agents, etc., may be used. The mixture of components may be added in any order that provides for the requisite binding.

In certain embodiments of this invention, the methods may further involve entering/recording test compounds that alter activity of Kvβ1.1 and/or an A-type K⁺ channel in a database.

Antibodies for use in the above-mentioned immunoassays are commercially available or can be easily prepared. Anti-Kvβ1.1 antibodies have been previously described by Veh et al. (1995) Eur. J. Neurosci. 7:2189-2205. Either polyclonal or monoclonal antibodies (anti-Kvβ1.1 antibodies) may be used in the immunoassays of the invention. Those of skill in the art will know of various techniques common in the immunology arts for purification and/or concentration of polyclonal antibodies, as well as monoclonal antibodies. See, for example, Coligan, et al. (1991) Unit 9, Current Protocols in Immunology, Wiley Interscience).

In the preferred embodiments of the invention, the immunoassays employ monoclonal antibodies (“mAb's”). For preparation of monoclonal antibodies, immunization of a mouse or rat is preferred. The term “antibody” as used in this invention includes intact molecules as well as fragments thereof, such as, Fab and F(ab′)^{2′}, and/or single-chain antibodies (e.g. scFv) which are capable of binding an epitopic determinant. The general method used for production of hybridomas secreting mAbs is well known. Confirmation of specificity among mAb's can be accomplished using relatively routine screening techniques (such as the enzyme-linked immunosorbent assay, or “ELISA”, BiaCore, etc.) to determine the binding specificity and/or avidity of the mAb of interest.

In addition, antibody fragments and human antibodies can be produced. Antibody fragments such as single chain antibodies (scFv or others), can be produced/selected using phage display technology. Human antibodies can be produced without prior immunization by displaying very large and diverse V-gene repertoires on phage, to generate libraries with innumerable antibodies against haptens, polysaccharides, self proteins, cell surface antigens, etc.

III. Functional Assays

In one embodiment, this invention provides methods of screening for agents that modulate Kvβ1.1 activity and hence performance in anxiety related tasks. The methods involve detecting the activity level of a Kvβ1.1 gene product (e.g. Kvβ1.1 subunit protein) in the presence of the compound(s) in question and/or comparing the activity level to controls such as KI or KO gene products. In particular, KI transgenic mammals may be used as a positive control to identify potential anxiolytic compounds. Examples of such approaches are described below.

A. Behavioral Assays

In one embodiment, modulators of Kvβ1.1 activity can be assayed using any of a variety of behavioral assays to test cognitive abilities, anxiety or stress responses. Such assays involve administering the test compound(s) to a WT, KI or KO mammal and then evaluating the effect of the test compound on the behavior of the mammal. Preferred behavioral assays measure cognitive, hippocampus-related tasks. Such assays include, but are not limited to contextual conditioning (see, e.g., Kim and Fanselow (1992) Science, 256:675; and Phillips and LeDoux Behav. Neurosci., 106:274), spatial learning (see, e.g., Morris et al. (1982) Nature, 297:681), rotor rod assay, conditioned taste aversion, social recognition, and the social transmission of food preferences (Bunsey and Eichenbaum (1995), Hippocampus, 5:546), some of which are illustrated in the examples below. Preferred behavioral assays that measure anxiety include elevated zero maze, conflict assays and fear-potentiated startle assay.

Since behavior at a particular time can depend on the particular physiological state of the mammal (e.g. feeding regimen, reproductive state, amount of sleep, etc.) such assays are preferably performed with both positive and negative controls. The negative control will be a mammal that has been treated in the same manner as the “test” mammal, but without administration of the test compound (or with administration of a significantly lower dose). In a preferred embodiment, a positive control will include a mammal having a Kvβ1.1 knock-in mutation, most preferably a mouse. A test compound, or combination of test compounds, that induces a reduction of anxiety similar to characteristic behavior of the KI Kvβ1.1 mammal will be an agent capable of modulating Kvβ1.1 activity.

B. Physiological Assays

1. Hormonal Assays

In another embodiment of this invention, hormonal assays may be used to evaluate an animal's response to stress and to assess the efficacy of a test compound in reducing stress. In this embodiment, a test compound is assessed for efficacy by studying the production of stress-related hormonal indicators such as corticosterone or c-fos activation. Corticosterone secretion from the adrenal glands in mice is generally considered a reliable endocrine hallmark of stress. Animals secrete this hormone in response to stress such that corticosterone response in plasma can be detected within 5 minutes after onset of stress. This response is often altered in either magnitude or time of secretion in animals that exhibit reduced anxiety levels. Animals are typically subjected to a mild form of stress invoked by environmental stimuli. Foot-shock, forced swim, and restraint are some of the common environmental stresses that have been practiced in rodents.

In one preferred embodiment of this assay, KI, KO and WT mammals are subjected to an environmental stress and the levels of their stress-related hormones are measured at different time intervals after stress. Most preferably, the hormone is plasma corticosterone and measurements are performed at time intervals of 0, 30, 60 and 90 minutes after onset of stress. In another preferred embodiment, activation of c-fos may be measured via in-situ hybridization, the levels of which may be digitized and quantitated, as explained in Example 8 below.

The above hormonal assays may furthermore be modified to include test compounds. In a most preferred embodiment, the subject animal is administered a test compound and after sufficient time to metabolize the test compound, the animal is subjected to an environmental stress. Measurement of the hormone level is then performed to determine whether the animal has a hormonal pattern that is consistent with the characteristic profile of a WT, KI or KO Kvβ1.1 mammal. Most preferably, test compounds are sought in which administration in a WT mammal produces a hormonal pattern consistent with an untreated KI mammal. As described in Example 5 below, transgenic KI mice have corticosterone levels which suggest a reduced anxiety pattern.

2. Hyperthermia Assays

Other assays may be used to evaluate an animal's response to stress and to assess the efficacy of a test compound for reducing stress. Stress-induced hyperthermia, as per the methods of Borsini et al. (1989) and Van der Heyden et al. (1997), can be used in conjunction with WT, KI and KO mice to evaluate their anxiety profile. Borsini et al. Psycholopharmacology (1989) 98:207-211, and Van Der Heyden, (1997) Physiology and Behavior 62:463-470. The animals are typically subjected to a mild form of environmental stress, typically a rectal probe is used for measuring the animal's temperature. As with the hormonal levels described above, the degree of an animal's hyperthermia in response to the environmental stress is an indication of the animal's anxiety.

As illustrated in Example 6 below, KI, KO and WT transgenic mammals are subjected to an environmental stress and the degree of hyperthermia is measured. Known anxiolytics, such as chlordiazepoxide, may also be used for comparative exercise to confirm whether the animal's responses are indeed consistent with an anxiolytic response. The hyperthermia assay may also be modified to assess the efficacies of test compounds, where the subject animal is administered a test compound and after sufficient time to metabolize the test compound, is subsequently subjected to an environmental stress. The degree of hyperthermia is then measured to determine whether the animal has a pattern of hyperthermia that is consistent with the characteristic profile of a WT, KI or KO Kvβ1.1 mammal. Most preferably, administration of the test compound to a WT mammal produces a pattern of hyperthermia consistent with an untreated KI mammal. As described in Example 6 below, stress-induced hyperthermia is blunted in KI mice to a similar magnitude in WT mice administered with chlordiazepoxide, providing further suggestion of a reduced anxiety pattern and therefore, an anxiolytic profile.

3. Electro-Physiological Assays

Kvβ1.1 activity can be readily measured by a variety of electro-physiological methods known in the art. As explained in work by Giese and colleagues, knockout of Kvβ1.1 activity results in both spike broadening during repetitive firing and a decrease in the slow after hyperpolarization (sAHP) of hippocampal CA1 neurons. Thus, the WT, KI or KO animals can be administered a test compound and then tested, or their tissue can be tested, to compare their electro-physiological response. Most preferably, an electrophysiological recording of the hippocampus is made, and more preferably a recording of the hippocampal CA1 neurons is made. Such a recording can be performed in vivo, however, in a preferred embodiment such recordings are made from hippocampal slice preparations. Methods of making and recording from such preparations are well known to those of skill in the art, as indicated in PCT Publication No. WO 00/24871 which is herein incorporated by reference. Preferably, test compounds are sought in which their administration in a WT mammal produces an electro-physiological pattern consistent with that of a KI mammal.

In addition, heterologous A-type K⁺ channels can be expressed in heterologous expression systems such that cells expressing knock-in Kvβ1.1 and corresponding Kv1 family α subunits can be measured electrophysiologically. Preferably the cells are Xenopus oocytes or HEK cells transfected with vector(s) containing the knock-in Kvβ1.1 and Kv1 α or their respective RNAs. These cells can be contacted with test compound(s) and the effect of the test compound(s) on the on A-type channel conductance can be measured and compared by whole-cell voltage clamp, current clamp, and/or with a patch-clamp technique. These methods for protein expression and electrophysiological recording are all well known to those skilled in the art.

4. Knock-in Kvβ1.1 Mammals as Positive Controls

As indicated above, the Kvβ1.1 knock-in mammals of this invention are useful in a wide variety of contexts, particularly as positive anxiolytic controls. High-level functions such as anxiety, are emergent neural network properties and as such cannot be assayed in highly reductionist (e.g. single synapse) models. Behavioral studies, for example, require an intact living mammal. Similarly, network-related properties (e.g. CA1 activity patterns) require a functional neural network.

Thus, evaluating the effects of potential modulators (e.g. test compounds) on such systems is greatly facilitated by the use of positive controls. In the present context, the behavior of a Kvβ1.1 knock-in mammal provides a good reference for evaluating the behavior of a “normal” mammal treated with a test compound. Other physiological indicators such as hormonal levels or hyperthermia may also be evaluated by comparison to a KI animal. Similarly the performance of a neurological preparation (e.g. the electrophysiological response of a hippocampal slice preparation) treated with a test compound can be compared to the performance of a neurological preparation obtained from a Kvβ1.1 knock-in mammal to evaluate the ability of the test compound(s) to alter electrophysiological response in a manner that mimics Kvβ1.1 down-regulation.

The KI mutants may also be crossed to other mouse models that show accelerated aging or accelerated brain degeneration (such as that found associated with Alzheimer's) to determine if the Kvβ1.1 knock-in modifies associated behavior disorders in these mammal models.

IV. Screening Kits

In one embodiment this invention provides kits for performing the assays described herein. The kits preferably include a Kvβ1.1 knock-in mammal or a cell or tissue derived from a Kvβ1.1 knock-in mammal. The kit can additionally include appropriate buffers and other solutions and standards for use in the assay methods described herein.

In addition, the kits may include instructional materials containing directions (i.e., protocols) for the practice of the methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

The following examples are carried out using standard techniques, which are well known and routine to those of skill in the art, except where otherwise described in detail. The following examples are presented for illustrative purpose, and should not be construed in any way limiting the scope of this invention.

### Example 1

### Generation of Kvβ1.1 Knock-In Mice Lacking the N-Terminus Necessary for Rapid Inactivation

Genetically-modified mice bearing a mutant Kvβ1.1 gene were developed that lack the ability to inactivate Kv1-family K⁺ channels but retain the ability to co-associate with Kv1 family α-subunits and thereby enhance channel surface expression. Unlike Kvβ1 gene knockouts, knock-in mice should retain all functional properties of Kvβ1.1 except the ability to confer N-type inactivation.

A. Generation of Kvβ1.1-Targeted ES cells

FIG. 1A indicates the endogenous mouse Kvβ1 gene structure and exonic organization prior to introduction of an immunoreactive epitope tag. The three Kvβ genes (Kvβ1.1, 1.2 and 1.3) each share exon domains 3-15 at the C-terminus, but are alternately spliced at the 5′ end to encode unique N-terminal protein sequences (exons 1.1, 1.2 and 1.2). For designing the Kvβ1.1 targeting construct as shown in FIG. 1B, a genomic Bac clone was isolated from the 129/SvEv mouse strain by screening the genomic library using a DNA fragment encoding the N-terminal 90 amino acids of rat Kvβ1.1 protein (Research Genetics, Bethesda, Md.) encoded by Genbank Accession No. X70662. The Bac clone was mapped and a 7.2 Kb BamHI fragment was isolated. A 1.2 Kb EcoRI fragment containing exon 1.1encoding the first 35 amino acids of Kvβ1.1 protein was replaced by a hemagglutinin epitope tag (SEQ ID NO: 2), 3×HA by overlapping PCR strategy, resulting in a protein that is functionally null for channel inactivation (KvBo). A neomycin-resistance cassette flanked by LoxP sequence was inserted within an intron 100 bp downstream of exon 1.1a at the EcoRI site. This modified EcoRI fragment was sequence-verified and ligated back with 1.5 Kb EcoRI and 4.2 RI/BamHI fragments to form the targeting vector.

R1 embryonic stem (ES) cells (Nagy et al. (1993) Proc. Natl. Acad.Sci. USA 90:8424-8428) were electroporated with a BamHI-linearized Kvβ1.1 targeting vector. The cells were selected by G418 and 200 colonies were picked for Southern Blot analysis. Genomic Southern analysis described below yielded 3 clones that underwent homologous recombination to incorporate the 3×HA tag along with neoFloxP.

Chimeric mice were generated from ES clones #145 and #191 bearing the 3×HA and neoFloxP allele by injection into C57BI6 mouse blastocysts. Male coat-color chimeras of 80-90% from each clone were selected to breed further with 129/SvEv wild-type females to obtain F1 heterozygotes on 129/SvEv background. These offsprings were intercrossed to generate F2 homozygotes for analysis. Inclusion of neoFloxP within a critical intron fortuitously suppressed transcription through the KvB1 gene, thus rendering this allele as Knockout for Kvb1. A subset of animals was out-bred onto C57/bl6 strain for 5 generations prior to being sib-mated for homozygote synthesis. All experiments unless otherwise stated were conducted either in 129/SvEv background or in N5-C57/bl6 background.

B. Derivation of Knock-In Mice Bearing the N-terminal Null Mutation for Kvβ1.1-(Kvβo)

Pronuclei from female heterozygous Kvβo-neoFloxP mice were collected and microinjected with a plasmid encoding Cre-recombinase. In eutero recombination was aimed to excise the Neo-Flox cassette in offsprings, leaving behind the 3×HA mutation at the N-terminal of Kvβ1.1 (Kvβo) and a single copy of Lox-P sequence within the intron. Following oviduct transfer of the microinjected pronuclei into pseudopregnant females, newborn pups were analyzed for evidence of Cre-mediated excision of the neoFloxP cassette. PCR analysis of genomic DNA revealed that nearly 100% of the offsprings bearing the 3×HA tag underwent in vivo excision.

C. Genomic Southern Blot Analysis

Genomic DNA from ES clones were prepared by Proteinase K digest for 3 hours at 37° C. Following precipitation in isopropanol, DNA were resuspended in TE and restriction digested with BamHI. DNA fragments were resolved on a 0.8% agarose gel and transferred onto nylon membranes overnight. A 300 bp BamHI/EcoRI fragment upstream of the targeting construct was radiolabeled with P³²dCTP and used as probe. Presence of an internal BamHI within the 3×HA sequence permits identification of a targeted ES cell as a band hybridizing at 1.2 Kb instead of the 7.2 Kb that is derived from the wild-type Kvβ1 allele. Presence of neoFloxP was subsequently confirmed using a probe directed to neo selection cassette (TK-neomycin minigene). FIG. 1C indicates the BamHI/EcoRi probe identified a ˜7.0 Kb band for the wild type Kvβ1 allele, and additional ˜1.0 Kb bands for the three targeted ES cells (191, 145, 51).

D. PCR analysis of Kvβo (KI) and Kvβneo Flox (KO)

A pair of primers (Kvβneo primers) were used to specifically detect the presence of Neo-FloxP sequence within the mice genome. Forward primer 1 (SEQ ID NO: 3; 5′ TTGAAAGTGACTTAACTCAGCGC) corresponding to the 3′ exon-intron boundary of exon 1.1, and reverse primer 1 (SEQ ID NO: 4; 5′ GCTGACCGCTTCCTCGTGCTTTAC) specific to the Neo-sequence, were used to amplify a 400 bp fragment from animals bearing the KvβNeo-Flox alleles. Both homozygous and heterozygous KO animals were confirmed by this analysis.

A second pair of primers (Kvβo KI primers) employing forward primer 1 and reverse primer 2 (SEQ ID NO: 5; 5′GGCCACATCTTAAAGATCGCAC) corresponding to 30 bp downstream of neoFlox insertion, were used to distinguish the zygosity of offsprings bearing the WT, Kvβo KI or the Kvβo KO allele. An additional primer pair specific to c-fos was used as internal positive control (SEQ ID NO: 6; fosF primer: 5′; AGGAGGGAGCTGACAGATACACTCC and SEQ ID NO: 7; fosR primer: 5′CAAGGATGGCTTGGGCTCAGGGTCGT). PCR amplification proceeded using genomic DNA from tail biopsies (1:200 dilution of tail digested in Proteinase K) for 30 cycles at 94° C., 54° C. and 72° C. for 30, 30 and 60 seconds, respectively. A 4% agarose gel (NuSieve 3:1, BMA, Rockland, Me.) was employed to resolve the resultant bands. As shown in FIG. 1E, the Kvβo KI primers amplify a 230 bp fragment from the wild-type Kvβ1.1 allele but amplify a 260 bp fragment from the Kvβo KI allele due to one copy of Lox-P sequence. The Kvβo KO allele does not typically amplify due to the size of neo-FloxP sequence (1.4 Kb insert size) intervening between the two primers but scores positively using the Kvβneo primers.

E. RNase Protection Assay

RNA probe complementary to wild-type Kvβ exon 1.1 and 3×HA-Ex1 were synthesized by in vitro transcription using T7 RNA polymerase in the presence of P³²CTP. Total hippocampal RNA (5 μg) from Kvβo (KI), Kvβo Neo-Flox (KO), and Kvβ1.1 wild-type animals were hybridized and Rnase-resistant bands were resolved on 6% PAGE following the manufacturer's instructions (RPAIII kit, Ambion, Austin, Tex.). FIGS. 1D and 1E provide a diagrammatic illustration of the exonic structure of the two probes as directed to mutated Kvβo (FIG. 1D) versus WT Kvβ1.1 (FIG. 1E), and the RPA gel resulting from hybridization to the respective probes. The 3×-HA probe revealed a shorter protected band of 220 bp in WT resulting from the missing 3×-HA sequence at the N-terminus of normal Kvβ1.1 mRNA. The same probe generated a 420 bp band in both heterozygote (WT/KI) and homozygote (KI/KI) animals. Conversely, the probe to normal Kvβ1.1 mRNA indicated a fully protected fragment of 480 bp from WT mRNA, while two fragments (210 and 140) flanking the 3×HA sequence were protected in KI mRNA.

These data together confirmed the expression of mRNA encoding the 3×-HA mutation (Kvβo) in KI animals. Importantly, animals bearing the Flox-neo cassette (n+/n+) did not demonstrate specific bands from either probes. Thus the Kvβ+Neo animals were completely deficient in Kvβ1.1 expression and were KO for Kvβ1.1.

F. Western Blot Analysis

Antibodies for Kvβ1 and Kvβ2 were used as described previously (Rhodes et al., 1997, J. Neuroscience 17:7084-9). Anti-HA polyclonal antibody (BMB, Germany) was used at 1:2000. Anti-Synapsin polyclonal antibody (Chemicon, Temecula, Calif.) was used at 1:5000. Brain regions from mice homozygous for Kvβo (KI), Kvβo Neo-flox (KO), or wild-type Kvβ1.1 were dissected and homogenized in Triton lysis buffer containing Triton X100 and 1×complete protease inhibitor cocktail (BMB, Germany). Protein samples were resolved on SDS-PAGE and transferred to nitrocellulose paper. Immunoreactive bands on the Western blot were detected by chemiluminescent exposure on film (Hyperfilm, Amersham).

Western Blot analysis indicated that the homozygous KI animals expressed Kvβo mRNA and protein in anatomical regions predicted by endogenous Kvβ1 expression and at levels comparable to wildtype mRNA. Brain regions from the cortex, striatum, hippocampus, cerebellum, midbrain and thalamus were dissected and analyzed for specific immuno-reactivities, as shown in FIG. 1G. Protein extracts (˜20 μg/lane) from each tissue were run along with rat brain membrane (RBM) as positive control. Wild-type (WT) tissues from normal 129/SvEv mice were assayed for Kvβ1, Kvβ2, Synapsin 1 and HA. Tissues from Kvβo-neofloxP (KO) animals were assayed as in WT samples, except HA assay was not performed. Tissue from Kvβo animals were assayed specifically for HA expression only. Endogenous Kvβ1 protein was identified in all brain areas of WT, with higher levels observed in the hippocampus and striatum. Kvβo mutant protein expression closely mimicked the expression pattern of endogenous Kvβ1, as indicated by enhanced HA immunoreactivity in the hippocampus and striatum of Kvβo KI animals.

Western blot analysis confirmed the presence of the HA tag and also demonstrated a lack of expression of Kvβ1 in Kvβo KO mice when using antibodies to Kvβ1. In these KO mice, there were no apparent changes in Kvβ2 expression when compared to WT animals, implying that no compensatory changes were in effect. In addition, Nakahira et al. have shown the absence of the Kvβ1 N-terminus does not adversely affect association with Kv1 family α-subunits (Nakahira et al., J Biol Chem (1996) 271:7084-7089). Use of antibody for co-immunoprecipitation as previously described in Rhodes et al., J. Neurosci. (1996) 16:4846-4860, revealed correct association of HA-tagged Kvβo with Kv1 family α subunits in KI mice (data not shown).

### Example 2

### Effects of Kvβ1.1 Subunits Lacking N-Terminus on Spatial Learning in Genetically Modified Mice

The significance of the N-terminus in Kvβ1.1 subunits was determined by comparing the phenotype of transgenic knock-in mice to the phenotypes of both knock-out mice and wild-type mice (129/SvEv) (“WT”). Mice were developed as described in Example 1 above, wherein the Kvβ1.1 of knock-in mice (“KI”) lacked a functional N-terminus, but were otherwise capable of associating with α-subunits. The knock-out mice (“KO”) had mutations in which Kvβ1.1 was completely inactivated and thus lacked both the N-terminus and the ability to coassociate with α-subunits.

Spatial learning is a hippocampus-dependent cognitive skill that can be tested in a Y maze paradigm (Dellu et al., “A two trial memory task with automated recording: study in young and aged rats” Brain Res. (1992) 588:132-139). The Y-maze novelty procedure is a two-trial recognition task, based on place exploration in a Y-maze. Each experiment consists of two, 5-minute exploration periods separated by an inter-trial interval (ITI). During the first exploration period, one arm of the Y-maze is occluded and the subject is allowed 5 minutes to explore the two arms of the maze. A camera mounted above the maze, records activity, and the data are analyzed by a computer. Trial 1 is followed by an ITI ranging from 30 minutes to 4 hours, during which the animal is placed in a holding cage. During the second exploration period, all arms of the Y-maze are open, the rodent is placed in the original start arm and allowed 5 minutes to explore the maze. The percent of subjects entering the novel arm first, the number of entries into the novel and non-novel arm and the time spent in each arm during the first 2-minutes of the trial are recorded. Rodents have a natural tendency to enter and explore the novel arm (Dellu et al., Brain Res. (1992) 588:132-139).

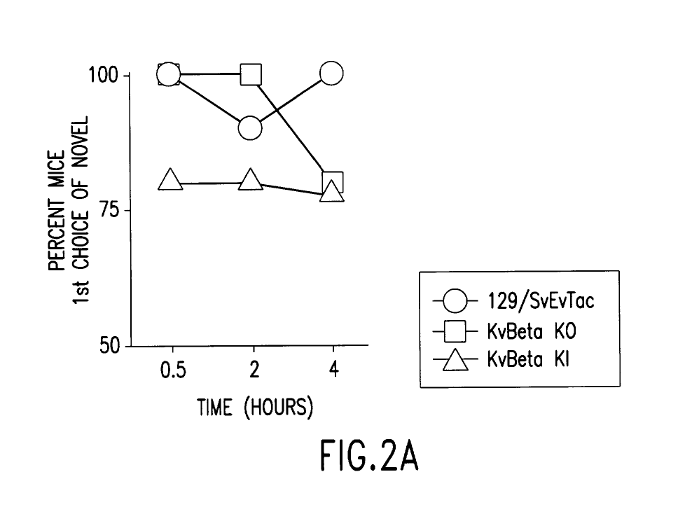

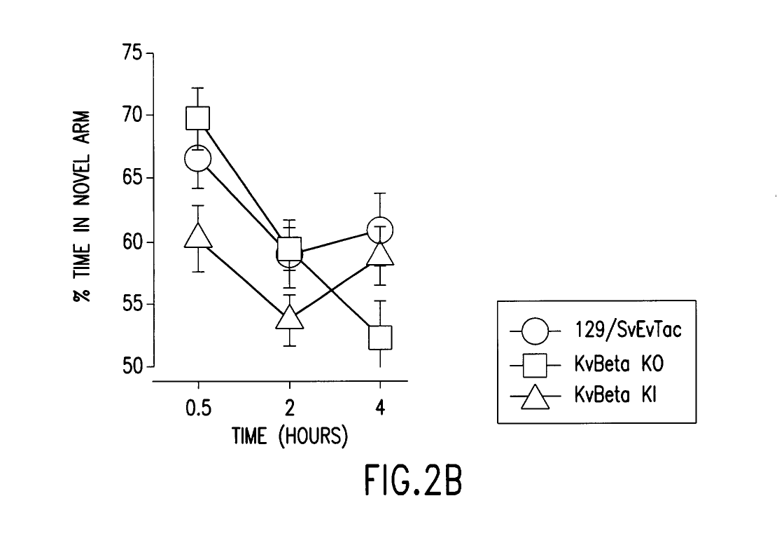

8-10 week old WT, KI and KO mice (n=28-30) in 129/SvEv background were tested in the Y-maze two trial place recognition procedure using different ITIs. All three WT, KI and KO groups spent significantly more time in the novel arm than in the non-novel arm during the retention trial at all ITIs tested (30 minutes, 2 hours and 4 hours). However, as shown in FIG. 2A, KO and WT mice consistently made a first choice of novel more frequently than KI mice at the 30 minute ITI. In contrast, only WT mice made a consistent first choice of the novel arm by the 4 hour ITI. This pattern suggest significant differences in recognition between the KI and KO mice at the 30 minute ITI that disappear by the 4 hour ITI. In addition, as indicated in FIG. 2B, KO and WT mice spent significantly more time in the novel arm (NA: 25.4±3.5 seconds) than KI mice at the 30 minute ITI (NA: 13.9±3.4 seconds, p<0.05). Conversely, KI and WT mice spent more time (approaching significance, p=0.055) in the novel arm than KO mice at the 4 hour ITI. That KI mice had retention deficits at the 30 minute ITI but better retention than KO mice at the 4 hour ITI suggests a complex role of Kvβ1.1 subunits in spatial learning tasks.

### Example 3

### Nociception and Physiological Responsiveness in Genetically Modified Mice

WT, KI and KO mice in 129/SvEv background were also assessed for differences in sensitivities to visual and nociceptive stimuli. WT, KI and KO mice were tested in groups of 8-10 per genotype for responsiveness to visual, tactile, thermal and chemical stimuli.

1. Visual Cliff Test

KI and KO mice were tested in groups of 8-10 per genotype in a visual cliff test to analyze differences in their visual depth perception (Fox, Animal Behavior (1965) 13(2): 232-233). The apparatus was a black and white (1″×1″ squares) checkered box (1′×2′×2′) contained within a larger box (2′×2′×2′8″) having an open top. The black and white checkered flooring extended across the top of the inner box, down the side and along the lower floor. A clear Plexiglass sheet (2′×2′) covered the top of the checkered inner box and completely covered the cliff. In the center of the box, a 4″×2.5″×1″ platform divided the cliff side from the safe side.

Mice were housed in groups until the day of experiment. The mouse was placed on the center platform and the latency of stepping off the platform as well as the side onto which the mouse stepped were recorded. If the mouse did not step off the platform within 3 minutes, a “no choice” score was noted and a maximum latency of 3 minutes was recorded. Each animal received one trial.

All KI and KO mice had 100% accuracy in the visual cliff task, suggesting that there were no differences in vision between groups.

2. Tactile Test using von Frey Filaments

WT, KI and KO mice were tested in groups of 8-10 per genotype for differences in tactile responsiveness using von Frey monofilaments (Chaplan et al., “Quantitative assessment of tactile allodynia in the rat paw,” J. Neurosci. Methods, (1994) 53:55-63).

Animals were placed on an elevated wire grid and the plantar surface of the paw was stimulated with a series of von Frey monofilaments. Von Frey filaments were applied to the mid-plantar hindpaw in sequential ascending or descending order, as necessary, to hover as closely as possible to the threshold of responses. The threshold was indicated by the lowest force that evoked a brisk withdrawal response to the stimuli. Thus, a withdrawal response led to the presentation of the next weaker stimulus, and a lack of withdrawal led to the presentation of the next stronger stimulus. Interpolation of the 50% threshold was calculated for each genotype.

3. Thermal and Chemical Test

WT, KI and KO mice in 129/SvEv background were tested in groups of 8-10 per genotype for differences in responsiveness to thermal and chemical stimuli by adapting tests from previous studies performed in rhesus monkeys (Negus et al., J Pharmacol Exp Ther 266:1355-1363, 1993; Brandt et al., J. Pharmacol Exp. Ther. 296(3): 939-946, 2001).

Warm water was used to assess differences in nociceptive responses produced by genetic manipulation. In the warm-water tail-withdrawal procedure, the rodent was gently restrained and the terminal (3 cm) end of the tail was placed into a thermos bottle containing water warmed to 42, 46, 50, 54 or 58° C. The latency for the animal to remove the tail from the water was recorded and used as a measure of nociception. If the animal did not remove the tail within 20 seconds, the experimenter removed the tail and a maximum latency was recorded.

Each experimental session began by determining mean baseline tail-withdrawal latencies in each temperature. These latencies were used to assess genotypes for potential differences in their sensitivity to the thermal stimuli.

Following baseline temperature determinations, capsaicin cream (0.075 mg % concentration) was applied to the entire length of the tail. Following 10 minutes, the tail was wiped with a damp cloth to remove excess cream and the temperature effect curve was redetermined 5 minutes later. This procedure has been shown to produce robust thermal-hypersensitive in mice.

Temperature-effect curves were generated for each experimental condition. In addition, the temperature that produced a half maximal increase in the tail-withdrawal latency (i.e., T₁₀) was calculated from each temperature-effect curve. The T₁₀ was determined by interpolation from a line drawn between the point above and the point below 10 seconds on the temperature-effect curve. Thermal hypersensitivity was defined as a leftward shift in the temperature-effect curve and a decrease in the T₁₀ value. Data were analyzed by analysis of variance (ANOVA) and significant main effects were analyzed further by post-hoc paired t-tests.

WT, KI and KO mice had similar responses to tactile (VonFrey filaments), thermal (warm water tail withdrawal) and chemical (capsaicin) stimuli. These data also suggest that Kv-channels associating with Kvβ1.1 are not involved in sensitivity to acute nociceptive stimuli.

### Example 4

### Mice Expressing Kvβ1.1 Subunits Lacking N-Terminus Display Impaired Contextual Fear Conditioning and Different Profiles on the Elevated Zero Maze

Previous work demonstrated that completely non-functional Kvβ1.1 knockout mice display decreased K-current inactivation and impaired performance in some hippocampal-formation dependent cognitive tasks, with no change in gross morphology or behavior (Giese et al. (1998) Learning & Memory 5:257-273). Thus the observed phenotype of the KO mice may be a result of the loss of the ability to 1) rapidly inactivate and/or 2) properly express the K-channel.

Group housed adult male mice were maintained under 12 hour light/dark cycle, with ad lib access to food and water. WT, KI and KO mice in both 129/SvEv and C57/BI6 background, aged 8-16 weeks, were separated into groups of 15-20 per genotype.

A. Contextual Fear Conditioning

Mice were allowed to habituate to the lab for 30-60 minutes prior to testing. Mice were then placed within a sound-attenuated enclosure (Med. Associates) equipped with a grid floor, white noise (64 dB), house lights, and tone (75 dB) or click generator, and were again allowed to habituate to the chamber for a brief period (2 minutes). Following the habituation period, a conditioning stimulus (CS) in the form of tone or a series of clicks, was presented for 30 seconds followed by a brief shock (2 seconds) (administered through a grid floor, 1.0 mAmp, unconditional stimulus, US). Following a 2 minute ITI, the animals were exposed to US and CS again. Thirty seconds after the second pairing of shocks, the mice were removed from the operant chambers. Approximately 18 hours later the mice were returned to the operant chambers and were observed for freezing behavior in response to the ‘context’ (i.e. the same environment, lighting, auditory stimuli other than CS). Following this observation period the animals were returned to their cages for 30-60 minutes. Following this delay, the mice were placed within a novel operant chamber and observed again for freezing behaviors. At the end of this observation period the CS was turned on and the freezing response was recorded. Freezing in each of these three conditions was monitored for 3 minutes in the 129/SvEv background animals and for 5 minutes in the C57BI6. At 10 second intervals the observer scored whether the animal is freezing (lack of all movement except respiration) or not. Percent freezing in each of the conditions was recorded for each animal. Data was analyzed using a two-way ANOVA (treatment x experimental condition) and post-hoc comparisons were made using Fisher's Least significant difference test.

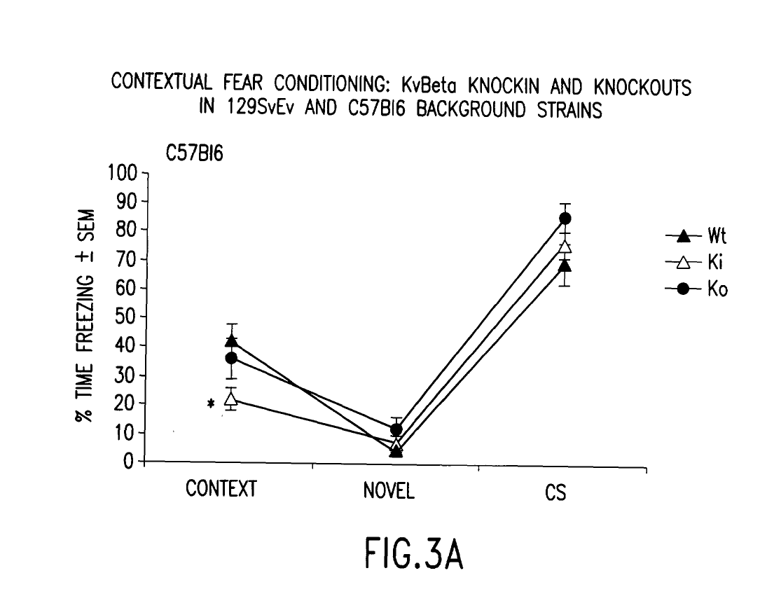

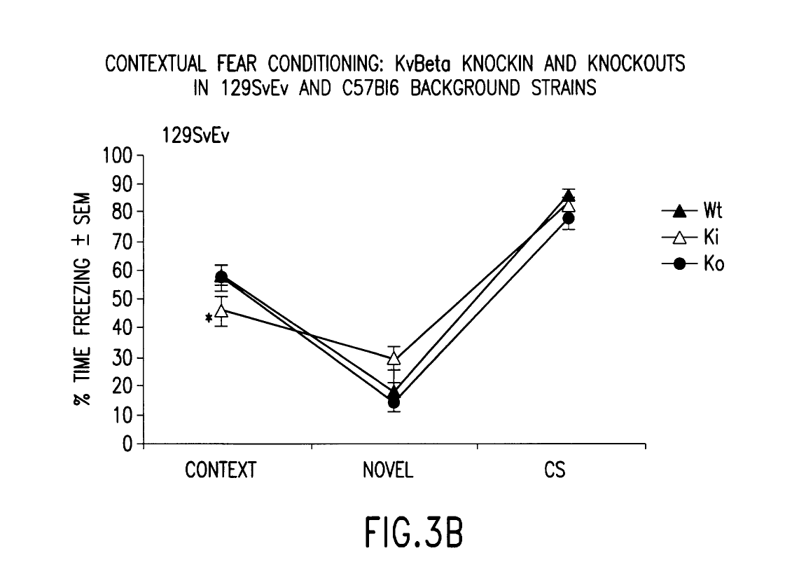

Replicating previously published work, no differences were observed between the KO and WT animals which both expressed high levels of freezing (better contextual conditioning). As shown in FIGS. 3A and 3B however, KI animals displayed significantly impaired levels of freezing (impaired contextual conditioning) compared to both KO and WT control animals, independent of the background mouse strain (p<0.05).

B. Elevated Zero Maze

Mice were allowed to habituate to an anteroom for at least 30 minutes. The zero maze consisted of a black Perspex circle divided into four equal quadrants (2 closed and 2 open). The closed quadrants have white Perspex walls extending up from the maze (inner wall=20 cm; outer wall=30 cm). The open quadrants have a clear Perspex lip that extends 3 mm up from the edge of the maze. The maze had an outer diameter of 60 cm and passages 5 cm in width, and was raised 55 cm off the floor. An individual animal was placed on the zero maze with head and forepaws in a closed quadrant. Experiments were run under red light conditions with one white light directed at the ceiling above the maze (C57/BI6, 15 watt, 4 lux, 129SvEv 42 watts, 20 lux). The mouse's behavior was then recorded for 4 minutes using the EthovisionPro video tracking system.

The amount of time spent in, and number of entries into, the open and closed quadrants as well as the total distance traveled was recorded by EthovisionPro software (Noldus Information Technology, Inc.). Data was analyzed with a one-way ANOVA and post-hoc comparisons were made using Fisher's Least significance difference test.

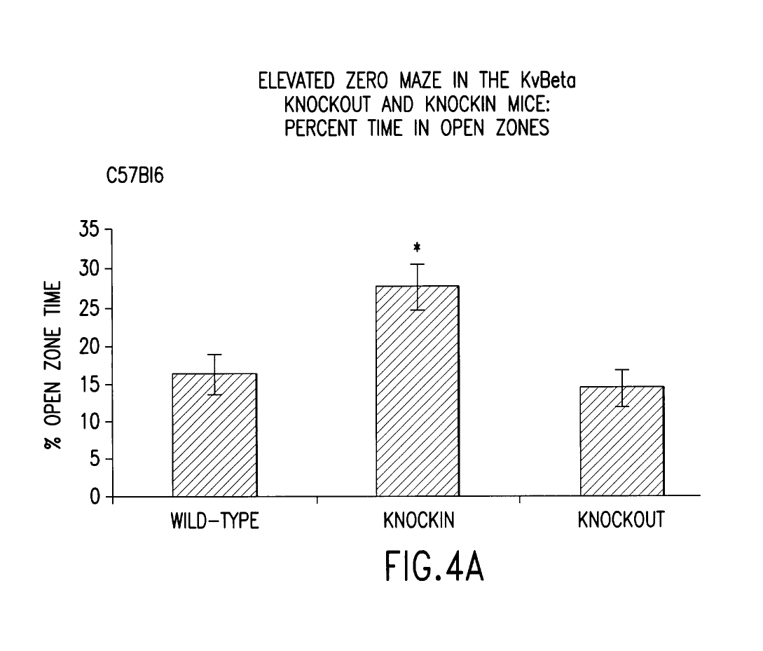

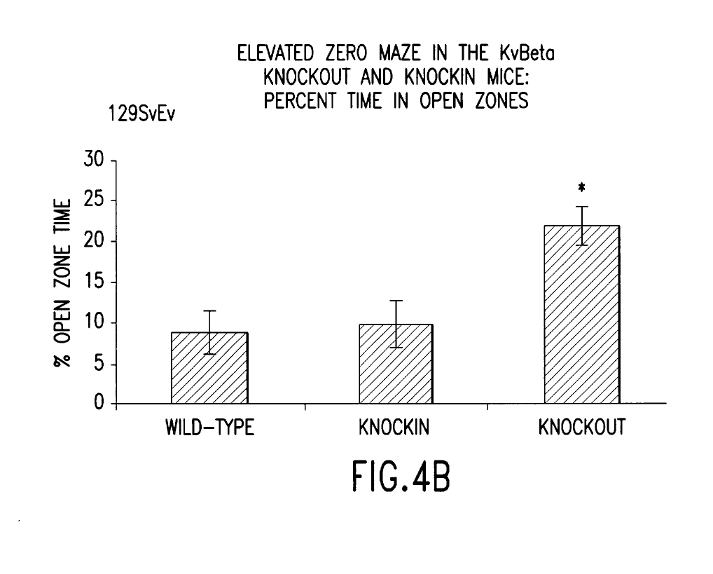

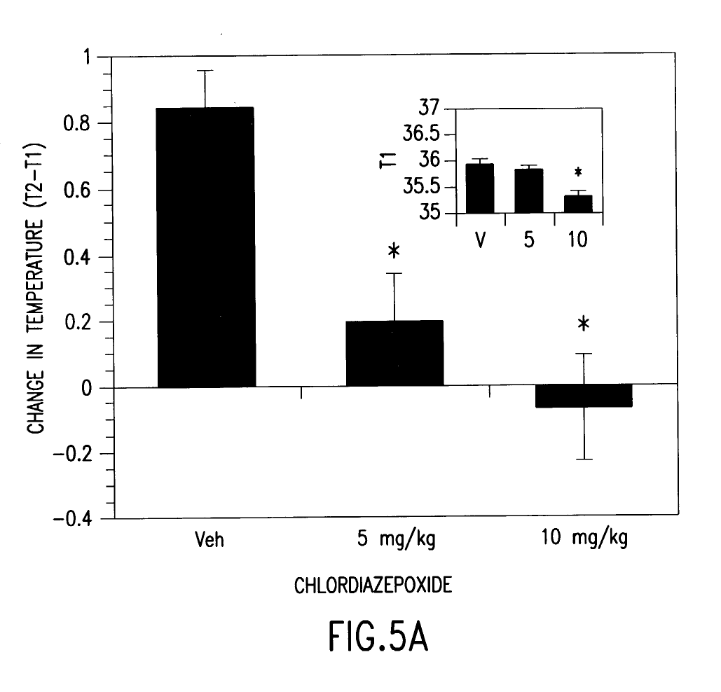

As in the contextual fear conditioning tests, KO and KI animals displayed different patterns of behavior in the elevated zero maze. FIG. 4A in particular indicates that mice homozygous for KI mutation in the C57BI6 background spent significantly more time (p<0.05) in the open zone as compared to KO or WT mice, suggesting a profile consistent with reduced anxiolytic activity. FIG. 4B illustrates that in 129/SvEv background animals, KO animals spent increased time in the open zone relative to both WT and KI animals. The different profiles between C57BI6 versus 129/SvEv background mice may be due to a generally lower level of exploration that is characteristic of 129/SvEv mice, which tend to remain in the quadrants in which they are first placed (closed quadrant). By comparison, as shown in FIGS. 4A and 4B, all C57BI6 mice, including WT, explore the environment to a greater extent and spent 5-20% more time in the open zone than 129/SvEv mice.

These data suggest, in combination with the Y maze studies of Example 2, that the phenotype observed in the KO mice is not due solely to the loss of the rapid inactivation of the K-channel and may reflect changes in K-channel expression or compensatory mechanisms involving other K-channel subunits.

### Example 5

### Effect of Knock-In Kvβ1.1 Subunits on Anxiety as Indicated by Stress-Induced Corticosterone Levels

To confirm whether the KI genotype provided a distinct anxiety profile, WT, KI and KO mice of 129/SvEv background were subjected to an environmental stress and then measured for changes in corticosterone levels as per the protocol of Kalman et al., Psychoneuroendocrinology (1994) 28(5): 349-360. In this experiment, restraint in a narrow, ventilated tube served as the environmental stress. WT, KI and KO mice were placed within the ventilated tube such that mobility was restrained for a period of 1 hour. Plasma corticosterone levels were measured at 0, 30, 60 minutes during stress, and additionally at 90 minutes after the onset of treatment (30 minutes post-restraint) to ascertain both the magnitude and the duration of corticosterone response in plasma. Preliminary results suggested that KI animals exhibited significantly reduced levels of corticosterone during this treatment (data not shown) (p<0.05).

### Example 6

### Effect of Knock-In Kvβ1.1 Subunits on Anxiety as Indicated by Measuring Stress-Induced Hyperthermia

Stress-induced hyperthermia, as per methods of Borsini et al (1989) and Van der Heyden et al. (1997) was used as an additional tool to evaluate the anxiolytic profile of a KI mouse (Borsini et al., Psycholopharmacology (1989) 98:207-211, and Van Der Heyden, Physiology and Behavior (1997) 62:463-470). This model has been reported to be sensitive to the effects of anxiolytics that prevent stress-induced hyperthermia.

Group housed adult male mice were maintained under 12 hour light/dark cycle, with ad lib access to food and water. WT, KI and KO mice aged 8-11 weeks in 129/SvEv background were separated into groups of 10 per genotype.

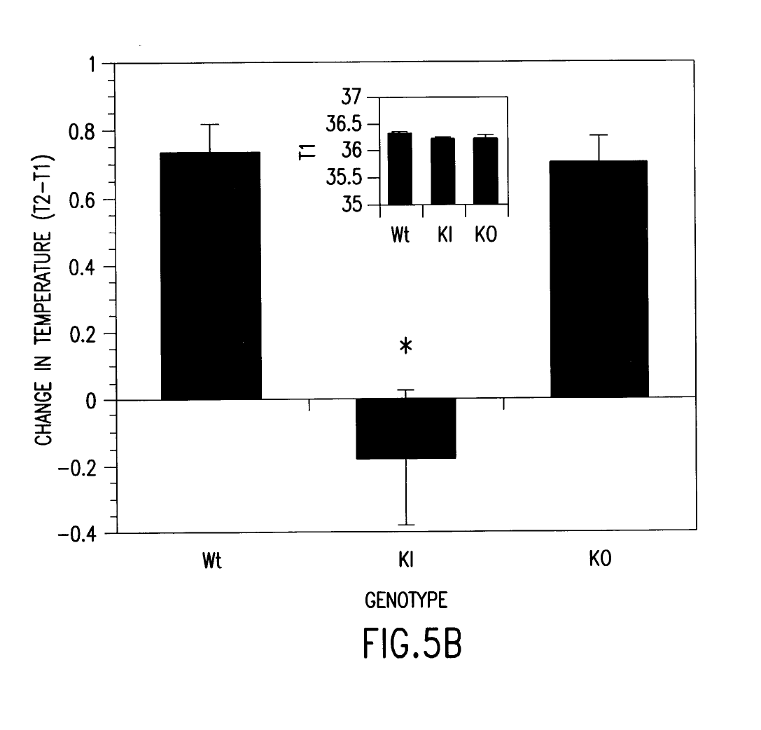

Mice were allowed to habituate to the test room at least 1 hour prior to testing. Mice were treated intraperitoneally with a control placebo vehicle (a placebo) or the anxiolytic chlordiazepoxide (5 or 10 mg/kg). Sixty minutes after injection, an initial core body temperature measurement (T1) was measured by inserting a lubricated thermistor probe 2 cm into the rectum of the mouse which was held under light restraint. In this experiment, the rectal probe served as the environmental stress. The temperature was read to the nearest 0.1 degree C. using a digital thermometer (Yellow Springs Instruments YSI 2100 Tele-thermometer). A second measurement (T2) was made 10 minutes later after the initial rectal probe. As indicated in FIG. 5A, control mice injected with the vehicle showed an increase in body temperature of approximately 0.7-0.8° C. in this procedure. Data was analyzed by one-way ANOVA followed by least significant difference tests (p<0.05).

FIG. 5A illustrates that the anxiolytic chlordiazepoxide prevented the stress-induced hyperthermia as compared to the control vehicle. In particular, the dosage level of 10 mg/kg chlordiazepoxide induced a drop in recorded temperature. Furthermore, as shown in FIG. 5B, untreated KI mice (n=25), subjected to the same procedure but in their own housing room, exhibited an anxiolytic-like response that was comparable to the mice treated with 10 mg/kg chlordiazepoxide. By contrast, untreated KO (n=16) and WT mice (n=20) displayed the same or expected hyperthermia as the control mice. No difference was observed in baseline (T1) temperature. These data provide further evidence that reduction of the inactivation of the Kv1 potassium channel, as modeled by the KI animal, would produce an anxiolytic effect.

### Example 7

### Seizure Threshold In Kvβ1 Mice

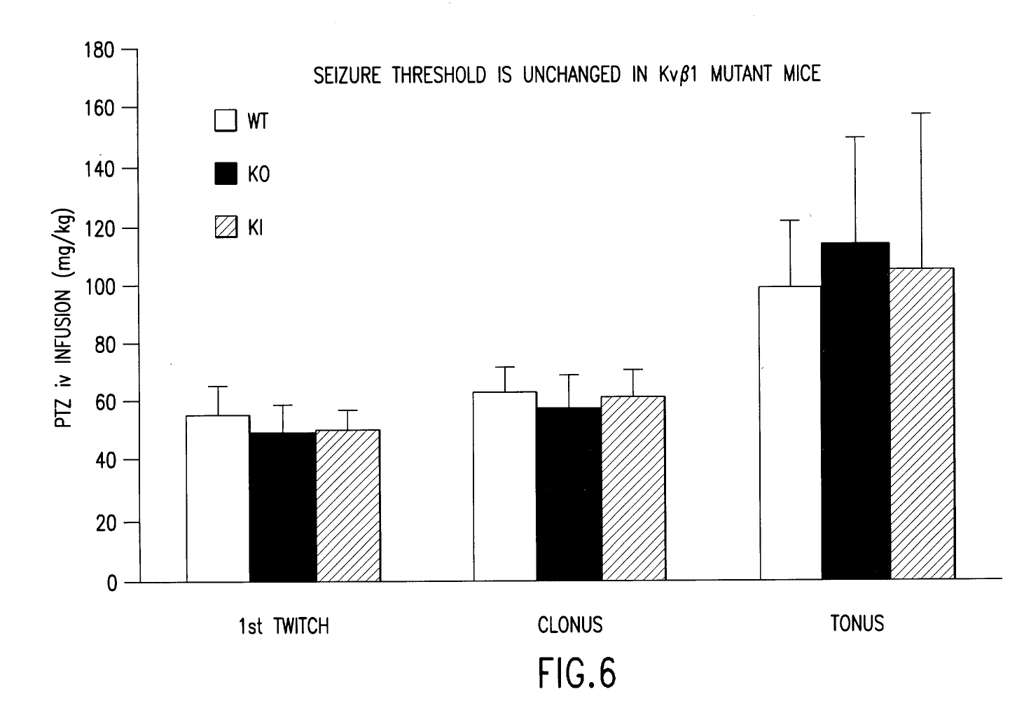

Mice underwent surgery under halothane anesthesia to implant a cannula (PE 10 tubing) into the external jugular vein. Animals were allowed to recover for at least 48 hours before seizure thresholds were conducted. Seizure thresholds were determined by administration of pentylenetatrazol (PTZ; 5 mg/ml in heparinized saline) delivered intravenously at a flow rate of 0.34 ml/minute. Mice were unrestrained but confined to a small chamber for observation. The latencies to the first twitch, clonic seizure (defined as rearing with forelimb padding), and tonic seizure (defined as full hindlimb extension) were recorded for each subject. Data on seizure latency were converted to mg/kg PTZ delivered for each of the behavioral endpoints as shown in FIG. 6.

Sensitivity to intravenous fusion of PTZ was identical among the three genetic strains of mice (n=10-12). Minimum infusion dose to elicit each of the responses shown in FIG. 6 were plotted.

### Example 8

### Stress Assessment Measuring c-fos Activation

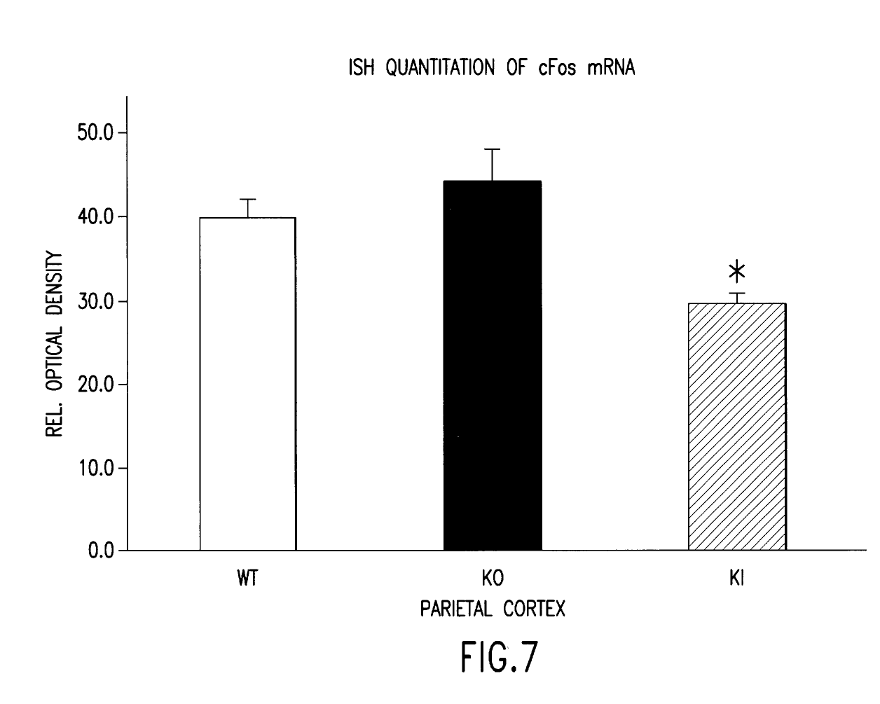

Wild-type, KI and KO mice were placed within a narrow enclosure (50 ml tube vented at one end) such that mobility was restrained for a period of 1 hour. Animals were restrained for 1 hour per day for 5 consecutive days and sacrificed immediately thereafter; fresh-frozen brains were processed for in-situ hybridization histochemistry. The C-terminal region of c-fos was PCR-cloned and used to synthesize a cDNA riboprobe in the presence of P³³UTP (SEQ ID NO: 8, forward primer 5′ AGGAGGGAGCTGACAGATCACTTC; SEQ ID NO: 9, reverse primer 5′ GTCTGCTGCATAGAAGGAACCGG). c-fos mRNA levels were increased in all three groups over unstressed controls c-fos. mRNA signal in the parietal cortex was digitized and quantitated as shown in FIG. 7. KI animals exhibited a 27% reduction in c-fos mRNA levels (p<0.05 by ANOVA; n=7 per group).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A transgenic rodent, said rodent comprising an endogenous gene cluster encoding a mutated Kvβ1.1 subunit of an A-type potassium channel, wherein the mutated Kvβ1.1 subunit is a knock-in subunit which is unable to confer N-type inactivation of the channel but retains the ability to co-associate with Kv1 family α-subunits.

2. The transgenic rodent of claim 1, wherein said rodent is a mouse.

3. The transgenic rodent of claim 1, wherein said knock-in subunit is encoded by a homozygous mutation.

4. The transgenic rodent of claim 1, wherein said knock-in subunit is encoded by a mutation selected from the group consisting of replacement mutations, insertion mutations, frameshift mutations, and stop codon mutations.

5. The transgenic mouse of claim 2, wherein said mouse has a significantly different learning or memory pattern as compared to a mouse of the same strain having a completely non-functional knock-out Kvβ1.1 subunit, as assayed by a Y maze.

6. The transgenic mouse of claim 5, wherein said mouse has significantly improved learning or memory after a 4 hour inter-trial interval as compared to the mouse having a knock-out Kvβ1.1 subunit.

7. The transgenic mouse of claim 5, wherein said mouse has significantly impaired learning or memory after a 30 minute inter-trial as compared to the mouse having a knock-out Kvβ1.1 subunit.

8. The transgenic mouse of claim 2, wherein said mouse has a significantly impaired learning pattern as compared to a mouse of the same strain having a knock-out Kvβ1.1 subunit, as assayed by contextual fear conditioning.

9. The transgenic mouse of claim 8, wherein said mouse has a significantly impaired learning pattern as compared to a mouse of the same strain having a wild-type Kvβ1.1 subunit, as assayed by contextual fear conditioning.

10. The transgenic mouse of claim 2, wherein said mouse has a significantly reduced anxiety pattern as compared to a mouse of the same strain having a knock-out Kvβ1.1 subunit, as assayed by the elevated zero maze.

11. The transgenic mouse of claim 10, wherein said mouse has a significantly reduced anxiety pattern as compared to a mouse of the same strain having a wild-type Kvβ1.1 subunit, as assayed by the elevated zero maze.

12. The transgenic mouse of claim 2, wherein said mouse has a significantly reduced anxiety pattern as compared to a mouse of the same strain having a knock-out Kvβ1.1 subunit, as assayed by stress-induced corticosterone levels.

13. The transgenic mouse of claim 12, wherein said mouse has a significantly reduced anxiety pattern as compared to a mouse of the same strain having a wild-type Kvβ1.1 subunit, as assayed by stress-induced corticosterone levels.

14. The transgenic mouse of claim 2, wherein said mouse has a significantly reduced anxiety pattern as compared to a mouse of the same strain having a knock-out Kvβ1.1 subunit, as assayed by stress-induced hyperthermia.

15. The transgenic mouse of claim 14, wherein said mouse has a significantly reduced anxiety pattern as compared to a mouse of the same strain having a wild-type Kvβ1.1 subunit, as assayed by stress-induced hyperthermia.

16. The transgenic mouse of claim 2, wherein said mouse has a significantly reduced anxiety pattern as compared to a mouse of the same strain having a knock-out Kvβ1.1 subunit, as assayed by stress-induced c-fos levels.

17. The transgenic mouse of claim 16, wherein said mouse has a significantly reduced anxiety pattern as compared to a mouse of the same strain having a wild-type Kvβ1.1 subunit, as assayed by stress-induced c-fos levels.

18. A transgenic rodent whose genome comprises a homozygous knock-in mutation in codons 1-70 of the N-terminus of an endogenous Kvβ1.1 subunit gene, wherein the knock-in mutation is a replacement mutation and the rodent exhibits significantly different cognitive patterns over a second rodent whose genome comprises a homozygous knock-out mutation which encodes a completely non-functional Kvβ1.1 subunit.

19. The transgenic rodent of claim 18, wherein the homozygous knock-in mutation is in codons 1-36 of the N-terminus of an endogenous Kvβ1.1 subunit gene.

20. The transgenic rodent of claim 19, wherein the replacement mutation comprises an immunoreactive epitope tag.

21. The transgenic rodent of claim 20, wherein the epitope tag is a hemagglutinin epitope tag.

22. The transgenic rodent of claim 18, wherein the rodent is a mouse.

23. A transgenic rodent whose genome comprises a homozygous knock-in mutation in codons 1-36 of the N-terminus of an endogenous Kvβ1.1 subunit gene, wherein the knock-in mutation is a replacement mutation and the rodent exhibits significantly different cognitive patterns over a second rodent whose genome comprises a homozygous knock-out mutation which encodes a completely non-functional Kvβ1.1 subunit.

24. A transgenic rodent all of whose germ cells and somatic cells contain a recombinant activated Kvβ1.1 transgene sequence introduced into said rodent or an ancestor of said rodent, at an embryonic stage, wherein the Kvβ1.1 transgene encodes a knock-in β subunit which is unable to confer N-type inactivation of a potassium channel but retains the ability to co-associate with Kv1 family α-subunits.

25. A method of making an isolated knock-in mammalian cell comprising the steps of:
(1) effecting homologous recombination between an endogenous Kvβ1.1 gene and a transgene Kvβ1.1, wherein said transgene Kvβ1.1 comprises
(a) a sequence encoding an immunoreactive tag substituting all or a portion of codons 1-70 of the Kvβ1.1 subunit,
(b) a selectable marker flanked by a pair of repeat sites, and
(c) a pair of sequences homologous to the endogenous Kvβ1.1 gene flanking both the tag and the selectable marker; and,
(2) effecting further recombination to remove the selectable marker, wherein the transgene Kvβ1.1 encodes a knock-in β subunit which is unable to confer N-type inactivation but retains the ability to co-associate with Kv1 family α-subunits.

26. A method of making an isolated knock-in mammalian cell comprising the steps of:
(1) effecting homologous recombination between an endogenous Kvβ1.1 gene and a transgene Kvβ1.1, wherein said transgene Kvβ1.1 comprises
(a) a sequence encoding an immunoreactive tag substituting all or a portion of codons 1-36 of the Kvβ1.1 subunit,
(b) a selectable marker flanked by a pair of repeat sites, and
(c) a pair of sequences homologous to the endogenous Kvβ1.1 gene flanking both the tag and the selectable marker; and,
(2) effecting further recombination to remove the selectable marker, wherein the transgene Kvβ1.1 encodes a knock-in β subunit which is unable to confer N-type inactivation but retains the ability to co-associate with Kv1 family α-subunits.

27. A mammalian cell expressing a mutated Kvβ1.1 subunit of an A-type potassium channel, wherein the mutated Kvβ1.1 subunit is a knock-in subunit which is unable to confer N-type inactivation of the channel but retains the ability to co-associate with Kv1α-subunits, wherein said cell comprises an endogenous nucleic acid sequence which controls expression of the mutated Kvβ1.1 subunit and said mutated Kvβ1.1 subunit is encoded by a mutation selected from the group consisting of a replacement mutation, an insertion mutation, a frameshift mutation, and a stop codon mutation.

28. The cell of claim 27, wherein said mutation is a replacement of all, or a portion of, codons 1-70 in the endogenous nucleic acid sequence.

29. The cell of claim 28, wherein said mutation is a replacement of all, or a portion of, codons 1-36 in the endogenous nucleic acid sequence.

30. A nucleic acid construct comprising a nucleic acid encoding a mutation in codons 1-70 of a Kvβ1.1 gene; wherein said nucleic acid encodes a knock-in subunit of an A-type potassium channel and said knock-in subunit is unable to confer N-type inactivation of the A-type potassium channel but retains the ability to co-associate with Kv1 family α-subunits.

31. A nucleic acid construct comprising a nucleic acid encoding a mutation in codons 1-36 of a Kvβ1.1 gene; wherein said nucleic acid encodes a knock-in subunit of an A-type potassium channel and said knock-in subunit is unable to confer N-type inactivation of the A-type potassium channel but retains the ability to co-associate with Kv1 family α-subunits.

32. A nucleic acid construct for disrupting expression of an endogenous Kvβ1.1 gene via homologous recombination, said construct comprising an immunoreactive epitope tag replacing all, or a portion of, codons 1-70 of the Kvβ1.1 gene, a selectable marker and a pair of nucleic acid sequences flanking both the tag and the selectable marker, wherein said pair is homologous to a portion of the endogenous Kvβ1.1 gene.

33. A nucleic acid construct for disrupting expression of an endogenous Kvβ1.1 gene via homologous recombination, said construct comprising an immunoreactive epitope tag replacing all, or a portion of, codons 1-36 of the Kvβ1.1 gene, a selectable marker and a pair of nucleic acid sequences flanking both the tag and the selectable marker, wherein said pair is homologous to a portion of the endogenous Kvβ1.1 gene.

34. A method of pre-screening test compounds for modulators of Kvβ1.1 subunit activity, comprising the steps of
(a) contacting test compounds with a mutated Kvβ1.1 subunit; and
(b) selecting one of the test compounds which provides a detectable change in the activity of the mutated Kvβ1.1 subunit,
wherein the mutated Kvβ1.1 subunit is a knock-in subunit which is unable to confer N-type inactivation but retains the ability to co-associate with Kv1 family α-subunits.

35. A method of pre-screening test compounds for modulators of Kvβ1.1 subunit activity, comprising the steps of
(a) contacting the test compounds with a wild-type Kvβ1.1 subunit and a mutated Kvβ1.1 subunit; and
(b) selecting one of the test compounds which provides a detectable change in the activity of the wild-type Kvβ1.1 subunit but no detectable change in the activity of the mutated Kvβ1.1 subunit,
wherein the mutated Kvβ1.1 subunit is a knock-in subunit which is unable to confer N-type inactivation but retains the ability to co-associate with Kv1 family α-subunits.

36. A method of assessing the efficacy of a test compound for modulating the activity of a Kvβ1.1 subunit, said method comprising:
(a) contacting the test compound with a wild-type Kvβ1.1 subunit and a mutated Kvβ1.1 subunit; and
(b) detecting a change in activity of the wild-type Kvβ1.1 subunit but no change in activity of the mutated Kvβ1.1 subunit,
wherein the mutated Kvβ1.1 subunit is a knock-in subunit which is unable to confer N-type inactivation of a potassium channel but co-associates with Kv1 family α-subunits.

37. A method of assessing the efficacy of a test compound for inactivating A-type potassium channels, said method comprising:
(a) contacting a test compound with a wild-type Kvβ1.1 subunit and a mutated Kvβ1.1 subunit; and
(b) detecting a change in the activity of the wild-type Kvβ1.1 subunit but no change in the activity of the mutated Kvβ1.1 subunit;
wherein the mutated Kvβ1.1 subunit is encoded by a knock-in Kvβ1.1 gene sequence comprising a mutation in all or a portion of codons 1-70.

38. A method of assessing the efficacy of a test compound for inactivating A-type potassium channels, said method comprising:
(a) contacting a test compound with a wild-type Kvβ1.1 subunit and a mutated Kvβ1.1 subunit; and
(b) detecting a change in the activity of the wild-type Kvβ1.1 subunit but no change in the activity of the mutated Kvβ1.1 subunit,
wherein the mutated Kvβ1.1 subunit is encoded by a knock-in Kvβ1.1 gene sequence comprising a mutation in all or a portion of codons 1-36.


SEQUENCE LISTING (9 sequences)

SEQ ID NO: 1 - DNA, 210 bp, Mus musculus
atggaagtctccatagcctgcacagagcacaatttgaagagtcgaaatggtgaggaccgacttctgagcaagcagagctccaccgcccccaatgtggtgaacgcagcccgggccaaattccgcactgtcgctatcatcgctcgcagcctggggaccttcacccctcagcatcacatttctctcaaagagtccaccgcaaagcagactggc

SEQ ID NO: 2 - DNA, 210 bp, Mus musculus
atggtattttacccatacgatgttcctgactatgcgggctatccctatgacgtcccggactatgcaggatcctatccatatgacgttccagattacgctgctcagtgccgggccaaattccgcactgtcgctatcatcgctcgcagcctggggaccttcacccctcagcatcacatttctctcaaagagtccaccgcaaagcagactggc

SEQ ID NO: 3 - DNA, 23 bp, Artificial Sequence
  Primer sequence
ttgaaagtgacttaactcagcgc

SEQ ID NO: 4 - DNA, 24 bp, Artificial Sequence
  Primer Sequence
gctgaccgcttcctcgtgctttac

SEQ ID NO: 5 - DNA, 22 bp, Artificial Sequence
  Primer Sequence
ggccacatcttaaagatcgcac

SEQ ID NO: 6 - DNA, 25 bp, Artificial Sequence
  Primer Sequence
aggagggagctgacagatacactcc

SEQ ID NO: 7 - DNA, 26 bp, Artificial Sequence
  Primer Sequence
caaggatggcttgggctcagggtcgt

SEQ ID NO: 8 - DNA, 24 bp, Artificial Sequence
  Primer Sequence
aggagggagctgacagatcacttc

SEQ ID NO: 9 - DNA, 23 bp, Artificial Sequence
  Primer Sequence
gtctgctgcatagaaggaaccgg